(19) United States
(12) Patent Application Publication
(10) Pub. No.: US 2023/0265407 A1
(43) Pub. Date: Aug. 24, 2023

(54) HUMAN RECOMBINANT ACE2-FC MUTANTS THAT DECOUPLE ANTI-SARS-COV-2 ACTIVITY FROM CARDIOVASCULAR EFFECTS

(71) Applicant: Northwestern University, Evanston, IL (US)

(72) Inventors: Jing Jin, Evanston, IL (US); Pan Liu, Evanston, IL (US)

(21) Appl. No.: 18/004,104

(22) PCT Filed: Jul. 2, 2021
(86) PCT No.: PCT/US2021/070820

§ 371 Date: Jan. 3, 2023

Related U.S. Application Data
(60) Provisional application No. 63/065,458, filed on Aug. 13, 2020.
(60) Provisional application No. 63/047,741, filed on Jul. 2, 2020.

(51) Int. Cl. C12N 9/48; C07K 16/10; A61P 31/14

(52) U.S. Cl. CPC C12N 9/485; C12Y 304/17023; C07K 16/10; A61P 31/14; C07K 2317/94; C07K 2317/524; C07K 2317/526; C07K 2319/30

(57) ABSTRACT

Disclosed are compounds, compositions, and methods for treating and/or preventing infection by viruses that utilize the angiotensin converting enzyme 2 (ACE2) as a cellular receptor such as sudden acute respiratory syndrome coronavirus 2 (SARS-CoV-2). Particularly disclosed are recombinant ACE2-Fc fusion proteins and ACE2 variants that exhibit anti-SARS-CoV-2 binding activity and decouple anti-SARS-CoV-2 activity from side effects such as cardiovascular effects, where the fusion proteins and variants exhibit reduced ACE2 enzymatic activity.

### CROSS-REFERENCE TO RELATED PATENT APPLICATIONS

The present application claims the benefit of priority under 35 U.S.C. § 119(e) to U.S. Provisional Application No. 63/047,741, filed on Jul. 2, 2020, and U.S. Provisional Application No. 63/065,458, filed on Aug. 13, 2020, the contents of which are incorporated herein by reference in their entireties.

### BACKGROUND

The invention relates to compounds, compositions, and methods for treating and/or preventing infection by viruses that utilize the angiotensin converting enzyme 2 (ACE2) as a cellular receptor such as sudden acute respiratory syndrome coronavirus 2 (SARS-CoV-2). In particular, the invention relates to recombinant ACE2-Fc fusion proteins and ACE2 variant that exhibit anti-SARS-CoV-2 binding activity and decouple anti-SARS-CoV-2 activity from side effects such as cardiovascular effects, where the fusion proteins and ACE2 variants exhibit reduced ACE2 enzymatic activity.

### SUMMARY

Disclosed are variants of ACE2, pharmaceutical compositions comprising the variants of ACE2, and treatment and prevention methods for coronavirus infection in a subject in need thereof. The disclosed variants of ACE2 may include polypeptide fragments of ACE2 having ACE2 activity for binding to coronavirus and having reduced cardiovascular effects. The polypeptide fragments of ACE2 preferably comprise a portion of the ectodomain of ACE2 and one or more mutations that reduce the enzymatic activity of ACE2 and corresponding cardiovascular effects. Preferably, the variants of ACE2 are soluble. The disclosed variants of ACE2 may be formulated as pharmaceutical compositions and may be administered to a subject having or at risk for developing coronavirus infection in order to treat and/or prevent the coronavirus infection preferably without causing cardiovascular effects.

### BRIEF DESCRIPTION OF THE FIGURES

FIG. 1. Mutagenesis strategies for catalytic inactivation of ACE2. A. Schematic representation of chimeric ACE2-Fc. The ectodomain sequence from amino acid 19 to 740 of human ACE2 is fused to the N-terminus of Fc domain of human IgG1, which forms a dimer via disulfide bridges (lines). The overall plan was to make individual point mutations (denoted as stars) of the catalytic site to inactivate the ACE2 peptidase activity. B. Mutant ACE2-Fc proteins, each carries a single alanine substitution of a selected residue, were produced by HEK-293 cells. As predicted, the proteins ran above the 250 kDa marker under nonreducing condition. C. The cocrystal structure (PDB: 6MOJ) shows SARS-CoV-2 spike RBD binding of ACE2. ACE2 exists in a clam shell-like configuration holding a catalytic cleft between its proximal and distal lobes. A zinc ion resides within the proximal lobe of the cleft void. D. In an inhibitor (MLN-4760)-bound structure of ACE2 (PBD: 1R4L), the inhibitor induced a conformational change of the catalytic cleft to adapt a ‘closed’ configuration[41]. E. Three proximal lobe residues H374, H378 and E402 formed interactions with the zinc ion. F. The side chains of six proximal and distal lobe residues, E145, R273, H345, P346, D368 and H505 formed direct interactions with inhibitor MLN-4760.

FIG. 2. Substrate-dependent inactivation of ACE2-Fc peptidase among ACE2-Fc mutants. Three peptide substrates were tested in catalytic reactions with ten individual variants of ACE2-Fc (wild-type and 9 mutants). The reactions were carried out in two different ways. Left panels: the reactions were performed using a high amount of purified ACE2-Fc enzyme (100 ng) with varying concentrations of the substrates between 0.39 and 200 μM (x-axis). Right panels: a lower dose of 10 ng ACE2-Fc was incubated with a fixed amount of 2 nmole of Mca-APK(Dnp) or 10 nmole of AngII/Apelin-13. Reactions proceeded for a standard length of time of 20 min. A. Surrogate fluorogenic substrate Mca-AP↓K(Dnp) was tested (↓: cleavage site). ACE2-Fc peptidase activities were compared between wild-type and mutants. Seven out of the nine mutants showed a completely loss-of-activity. B. When AngII was used in reactions with the ACE-2-Fc panel, DRVYIHP↓F of AngII sequence was cleaved by ACE2, releasing a Phe/F. The assay detected the generation of amino acid Phe/F as the results of ACE2-Fc activity. C. Similar to AngII, Apelin-13 was cleaved by ACE2 between the proline(P)-phenylalanine (F) bond in its sequence, Pyr-RPRLSHKGPMP↓F. The rates of Phe/F release from the reactions were detected. Data are shown as the mean±SD from triplicate experiments.

FIG. 3. Binding affinities of individual ACE2-Fc variants to RBD of SARS-CoV-2 spike protein. A. A fixed amount of recombinant viral RBD protein was coated to an ELISA plate (buffer coated well were used as controls). Wild-type and nine mutants of ACE2-Fc were added to the wells at varying concentrations between 0.5 ng/mL and 1200 ng/mL (x-axis). Binding was determined by the difference in signal intensity between the RBD-coated and the corresponding control wells. B. While all variants of ACE2-Fc exhibited affinity to viral RBD protein, there were differences in their calculated EC₅₀ values. Data are shown as the mean±SD from triplicate experiments.

FIG. 4. Inhibition of a pseudotyped virus by wild-type ACE2-Fc and AACE2-Fc variants. A. The transduction activity of a pseudotyped virus expressing SARS-CoV-2 spike protein to HEK293 cells expressing receptor ACE2 was measured through a firefly luciferase reporter. The cell transduction assays were performed in the presence of various concentrations of individual ACE2-Fc variants. B. IC₅₀ values were calculated based on calculated ACE2-Fc concentrations needed to inhibit 50% reporter activity. Data are shown as the mean±SD from triplicate experiments.

FIG. 5. Pharmacokinetics of wild-type ACE2-Fc and AACE2-Fc variants. A. After a bolus i.v. injection of the listed ACE2-Fc variants in mice, drug concentration in blood was monitored over a period time. B. t_{(1/2)} values of individual biologics were calculated by GraphPad Prism software. Data are shown as the mean±SD (n=3).

FIG. 6. Summary of ACE2-Fc mutagenesis. A. Schematics of ACE2-Fc fusion construct: in an N-to-C-terminus order, ACE2-Fc is comprised of signal peptide and ACE2 ectodomain of human sequence, followed by Fc derived from human IgG1. B. The amino acid sequence of wild-type ACE2-Fc, showing signal peptide, ACE2 ectodomain and Fc with distinct font colors. The single amino acids in red fonts were individually mutated to alanine. C. List of the 9 mutants of ACE2-Fc.

FIG. 7. The consensus substrate motif of ACE2. A. ACE2 cleaves surrogate peptide of Mca-APK-(Dnp) between proline (P) and lysine (K) residues (arrow). B. ACE2 cleaves proline-phenylalanine (P-F) peptide bonds at the C-termini of its physiological peptides.

FIG. 8. Substrate-dependent activities among ACE2 mutants. (A) Surrogate substrate Mca-APK-DNP is cleaved between P and K as compared to the P-F cleavage site in Ang II by ACE2. (B) ACE2 activity assays were performed using either Mca-APK-DNP or Ang II as a substrate, with individual variants of ACE2-Fc as the enzyme. H345A showed loss of activity toward the surrogate (Left; N.D. for not detected), in agreement with the findings by Glasgow et al. (1). However, ACE2-Fc enzymatic activity measured by Ang II showed different results, with H345A having full activity as compared to wild-type ACE2-Fc (Right). Meanwhile, R273A showed loss of activity against Ang II. (C) To further confirm the specificity of the reaction, we conducted mass spectrometry analysis of the peptide(s) generated from the reactions, using Ang II peptide as substrate, that further confirmed the loss of activity by the R273A mutation, whereas H345A of ACE2 remained fully active.

FIG. 9. Inhibition of infection and cytotoxicity of SARS-CoV-2 by remdesivir and ACE2(273)-Fc. IC50 (Infection: Square (▪)) and CC50 (Cytotoxicity: Circle (●)).

FIG. 10. Viability and % Infection for cells treated with ACE2(273)-Fc as in FIG. 9.

FIG. 11. Inhibition of infection and cytotoxicity of SARS-CoV-2 by remdesivir and ACE2(273)-Fc. IC50 (Infection: Square (▪)) and CC50 (Cytotoxicity: Circle (●)).

FIG. 12. Viability and % Infection data for cells treated with ACE2(273)-Fc as in FIG. 11.

### DETAILED DESCRIPTION

The present invention is described herein using several definitions, as set forth below and throughout the application.

As used in this specification and the claims, the singular forms “a,” “an,” and “the” include plural forms unless the context clearly dictates otherwise. For example, the term “a polypeptide fragment” should be interpreted to mean “one or more a polypeptide fragment” unless the context clearly dictates otherwise. As used herein, the term “plurality” means “two or more.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

As used herein, the term “subject” may be used interchangeably with the term “patient” or “individual” and may include an “animal” and in particular a “mammal.” Mammalian subjects may include humans and other primates, domestic animals, farm animals, and companion animals such as dogs, cats, guinea pigs, rabbits, rats, mice, horses, cattle, cows, and the like.

The disclosed methods, compositions, and kits may be utilized to treat a subject in need thereof. A “subject in need thereof” is intended to include a subject having or at risk for developing diseases and disorders such as coronavirus infection such as an infection by sudden acute respiratory syndrome coronavirus 2 (SARS-CoV-2).

The terms “amino acid” and “amino acid sequence” refer to an oligopeptide, peptide, polypeptide, or protein sequence (which terms may be used interchangeably), or a fragment of any of these, and to naturally occurring or synthetic molecules. Where “amino acid sequence” is recited to refer to a sequence of a naturally occurring protein molecule, “amino acid sequence” and like terms are not meant to limit the amino acid sequence to the complete native amino acid sequence associated with the recited protein molecule.

The amino acid sequences contemplated herein may include one or more amino acid substitutions relative to a reference amino acid sequence. For example, a variant polypeptide may include non-conservative and/or conservative amino acid substitutions relative to a reference polypeptide. “Conservative amino acid substitutions” are those substitutions that are predicted to interfere least with the properties of the reference polypeptide. In other words, conservative amino acid substitutions substantially conserve the structure and the function of the reference protein. The following Table provides a list of exemplary conservative amino acid substitutions.

| Original Residue | Conservative Substitution |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain one or more of: (a) the structure of the polypeptide backbone in the area of the substitution, for example, as a beta sheet or alpha helical conformation, (b) the charge or hydrophobicity of the molecule at the site of the substitution, and/or (c) the bulk of the side chain. Non-conservative amino acid substitutions generally do not maintain one or more of: (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

A “deletion” refers to a change in a reference amino acid sequence (e.g., any of SEQ ID NOs:1-23) that results in the absence of one or more amino acid residues. A deletion removes at least 1, 2, 3, 4, 5, 10, 20, 50, 100, or 200 amino acids residues or a range of amino acid residues bounded by any of these values (e.g., a deletion of 5-10 amino acids). A deletion may include an internal deletion or a terminal deletion (e.g., an N-terminal truncation or a C-terminal truncation of a reference polypeptide). A “variant” of a reference polypeptide sequence may include a deletion relative to the reference polypeptide sequence. For example, SEQ ID NO:2 (amino acids 1-740) SEQ ID NO:3 (amino acids 1-619), and SEQ ID NO:4 (amino acids 1-555) include C-terminal deletions relative to reference sequence SEQ ID NO:1 (amino acids 1-805).

The words “insertion” and “addition” refer to changes in an amino acid sequence resulting in the addition of one or more amino acid residues. An insertion or addition may refer to 1, 2, 3, 4, 5, 10, 20, 30, 40, 50, 60, 70, 80, 90, 100, 150, or 200 amino acid residues or a range of amino acid residues bounded by any of these values (e.g., an insertion or addition of 5-10 amino acids). A “variant” of a reference polypeptide sequence may include an insertion or addition relative to the reference polypeptide sequence.

A “fusion polypeptide” refers to a polypeptide comprising at the N-terminus, the C-terminus, or at both termini of its amino acid sequence a heterologous amino acid sequence, for example, a heterologous amino acid sequence that extends the half-life of the fusion polypeptide in blood. A “variant” of a reference polypeptide sequence may include a fusion polypeptide comprising the reference polypeptide.

A “fragment” is a portion of an amino acid sequence which is identical in sequence to but shorter in length than a reference sequence (e.g., any of SEQ ID NOs:1-23). A fragment may comprise up to the entire length of the reference sequence, minus at least one amino acid residue. For example, a fragment may comprise from 5 to 1000 contiguous amino acid residues of a reference polypeptide. In some embodiments, a fragment may comprise at least 5, 10, 15, 20, 25, 30, 40, 50, 60, 70, 80, 90, 100, 150, 250, or 500 contiguous amino acid residues of a reference polypeptide; or a fragment may comprise no more than 5, 10, 15, 20, 25, 30, 40, 50, 60, 70, 80, 90, 100, 150, 250, or 500 contiguous amino acid residues of a reference polypeptide; or a fragment may comprise a range of contiguous amino acid residues of a reference polypeptide bounded by any of these values (e.g., 40-80 contiguous amino acid residues). Fragments may be preferentially selected from certain regions of a molecule. The term “at least a fragment” encompasses the full length polypeptide. A “variant” of a reference polypeptide sequence may include a fragment of the reference polypeptide sequence. For example, SEQ ID NO:2 (amino acids 1-740) SEQ ID NO:3 (amino acids 1-619), and SEQ ID NO:4 (amino acids 1-555) comprise fragments of reference sequence SEQ ID NO:1 (amino acids 1-805).

“Homology” refers to sequence similarity or, interchangeably, sequence identity, between two or more polypeptide sequences. Homology, sequence similarity, and percentage sequence identity may be determined using methods in the art and described herein.

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastp,” that is used to align a known amino acid sequence with other amino acids sequences from a variety of databases.

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 100, at least 150, at least 200, at least 250, at least 300, at least 350, at least 400, at least 450, at least 500, at least 550, at least 600, at least 650, or at least 700 contiguous amino acid residues; or a fragment of no more than 15, 20, 30, 40, 50, 100, 150, 200, 250, 300, 350, 400, 450, 500, 550, 600, 650, or 700 amino acid residues; or over a range bounded by any of these values (e.g., a range of 500-600 amino acid residues) Such lengths are exemplary only, and it is understood that any fragment length supported by the sequences shown herein, in the tables, figures or Sequence Listing, may be used to describe a length over which percentage identity may be measured.

In some embodiments, a “variant” of a particular polypeptide sequence may be defined as a polypeptide sequence having at least 20% sequence identity to the particular polypeptide sequence over a certain length of one of the polypeptide sequences using blastp with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of polypeptides may show, for example, at least 20%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length of one of the polypeptides, or range of percentage identity bounded by any of these values (e.g., range of percentage identity of 80-99%).

The disclosed methods of treatment and pharmaceutical composition utilize and/or include angiotensin converting enzyme 2 (ACE2) or variants thereof such as fragments of ACE2. The nucleotide sequence of the human ACE2 gene is available from the National Center for Biotechnology Information of the National Institutes of Health. The location of the human ACE2 gene is provided as NC 000023.11 (15494525..15602069, complement). ACE2, isoform 1, is a transmembrane protein which is expressed first as a precursor polypeptide having the amino acid sequence (SEQ ID NO:1).

Amino acids 1-17/18 are a leader peptide which is cleaved from mature ACE2. Amino acids 18/19-740 are extracellular. The fusion proteins and ACE2 variants disclosed herein may include or lack the natural leader peptide of ACE2 (amino acids 1-18 or amino acids 19 of SEQ ID NO:1). Alternatively, the fusion proteins and ACE2 variants may comprise a heterologous leader peptide.

Amino acids 741-761 form a helical transmembrane sequence. Amino acids 762-805 are cytoplasmic. Natural variants of ACE2 are contemplated herein and may include the natural variant K26R and the natural variant N638S. Natural isoforms of ACE2 also are contemplated herein include isoform 2 having the following differences relative to isoform 1: F555L and 4556-805. The variants of ACE2 disclosed herein, including fragments of ACE2, may have or lack one or more of these amino acid sequences of ACE2.

Fusion polypeptides of ACE2 or variants thereof are disclosed herein. The fusion polypeptide of ACE2 or a variant thereof may include the amino acid sequence of ACE2 or a variant thereof (e.g., the amino acid sequence of a fragment of ACE2) fused to a heterologous amino acid sequence. Preferably, the heterologous amino acid sequence increases the half-life of the fusion polypeptide in blood.

The disclosed fusion polypeptides may comprise the amino acid sequence of ACE2 or a variant thereof (e.g., the amino acid sequence of a fragment of ACE2) fused directly to a heterologous amino acid sequence or fused via a linker sequence. Suitable linker sequences may include amino acid sequences of 5, 6, 7, 8, 9, 10, 11, 12, 13, 14, or 15 amino acids or more, or a range bounded by any of these values (e.g., a linker of 5-15 amino acids). In some embodiments, the linker sequence comprises only glycine and serine residues.

Fusion polypeptides disclosed herein include the amino acid sequence of ACE2 or a variant thereof fused to the amino acid sequence of an antibody or to one or more fragments of an antibody, for example, the Fc portion of an antibody (constant fragment of human IgG (e.g., a fragment of the constant region of IgG comprising the hinge region, the CH2 region, and the CH3 region such as SEQ ID NO:14).

ACE2 is a carboxypeptidase which catalyzes the conversion of angiotensin I to angiotensin 1-9, a protein of unknown function, and catalyzes the conversion of angiotensin II (1-8) to angiotensin (1-7) (EC:3.4.17.23), which is a vasodilator. ACE2 also catalyzes the hydrolysis of apelin-13 and dynorphin-13. ACE2 also is the cellular receptor for sudden acute respiratory syndrome (SARS) coronavirus/SARS-CoV and human coronavirus NL63/HCoV-NL63. The variants of ACE2 disclosed herein, including fragments of ACE2, may have or lack one or more of these enzymatic activities of ACE2.

In catalyzing the conversion of angiotensin II (1-8) to angiotensin (1-7), ACE2 catalyzes the following reaction: angiotensin II (1-8)+H₂O=angiotensin (1-7)+L-phenylalanine, which removes the C-terminal phenylalanine of angiotensin II (1-8). ACE2 has cofactor binding sites for Zn2+ and Cl⁻. The Michaelis constants (Kₘ) for these reactions are as follows: Kₘ=6.9 μM for angiotensin I; Kₘ=2 μM for angiotensin II; Kₘ=6.8 μM for apelin-13; and Kₘ=5.5 μM for dynorphin-13. The optimum pH for these reactions is 6.5 in the presence of 1 M NaCl, but ACE2 is active at pH 6-9. ACE2 is activated by halide ions chloride and fluoride, but not bromide. ACE2 is inhibited by MLN-4760, cFP_Leu, and EDTA, but not by the ACE inhibitors linosipril, captopril and enalaprilat. The variants of ACE2 disclosed herein, including fragments of ACE2, may have or lack one or more of these enzymatic activities of ACE2. In some embodiments, the variants of ACE2 disclosed herein, including fragments of ACE2, may have a Michaelis constant for one or more of the reactions above which is ±50% of the Michaelis constant for ACE2.

The variants of ACE2 disclosed herein, including fragments of ACE2, may have biological activities that include binding to the coronavirus. For example, the variants of ACE2 disclosed herein may bind to the spike protein of coronavirus.

ACE2 exhibits molecular functions and enzymatic functions that may include: carboxypeptidase activity, endopeptidase activity, glycoprotein binding activity, metallocarboxypeptidase activity, cleavage of Angiotensin II, zinc ion binding activity, and binding to the coronavirus as a receptor for the coronavirus. The variants of ACE2 disclosed herein, including fragments of ACE2, may have at least one, but preferably all of the molecular and enzymatic functions of ACE2.

Key structure features of ACE2 and the variants of ACE2 disclosed herein may include one or more of the following: amino acid position 169—chloride binding site; amino acid position 273—substrate binding site; amino acid position 345 substrate binding site; amino acid position 346—substrate binding site via a carbonyl oxygen; amino acid position 371—substrate binding site; amino acid position 374—metal binding site (e.g., Zn21; amino acid position 375—active site; amino acid position 378—catalytic metal binding site (e.g. Zn²⁺); amino acid position 402—catalytic metal binding site (e.g. Zn²⁺); amino acid position 477—chloride binding site; amino acid position 481—chloride binding site; amino acid position 505—active site; and amino acid position 515 substrate binding site. The variants of ACE2 disclosed herein, including fragments of ACE2, may have or lack one or more of these structural features of ACE2.

Key structure features of ACE2 and the variants of ACE2 disclosed herein may include one or more of the following: amino acid positions 23-52—helix; amino acid positions 56-77; amino acid positions 78-82—turn; amino acid positions 85-87—helix; amino acid positions 91-100—helix; amino acid positions 104-107—helix; amino acid positions 110-129—helix; amino acid positions 131-134—beta strand; amino acid positions 137-143—beta strand; amino acid positions 144-146—turn; amino acid positions 148-154—helix; amino acid positions 158-171—helix; amino acid positions 173-193—helix; amino acid positions 196-198—beta strand; amino acid positions 199-204—helix; amino acid positions 205-207—turn; amino acid positions 213-215—turn; amino acid positions 220-251—helix; amino acid positions 253-255—turn; amino acid positions 258-260—beta strand; amino acid positions 264-266—helix; amino acid positions 267-271—beta strand; amino acid positions 279-282—helix; amino acid positions 284-287—turn; amino acid positions 294-297—turn; amino acid positions 298-300—helix; amino acid positions 304-316—helix; amino acid positions 317-319—turn; amino acid positions 327-330—helix; amino acid positions 338-340—beta strand; amino acid positions 347-352—beta strand; amino acid positions 355-359—beta strand; amino acid positions 366-384—helix; amino acid positions 385-387—turn; amino acid positions 390-392—helix; amino acid positions 400-413—helix; amino acid positions 415-420—helix; amino acid positions 422-426—turn; amino acid positions 432-446—helix; amino acid positions 449-465—helix; amino acid positions 466-468—beta strand; amino acid positions 473-483—helix; amino acid positions 486-488—beta strand; amino acid positions 499-502—helix; amino acid positions 504-507—helix; amino acid positions 514-531—helix; amino acid positions 532-534—turn; amino acid positions 539-541—helix; amino acid positions 548-558—helix; amino acid positions 559-562—turn; amino acid positions 566-574—helix; amino acid positions 575-578—beta strand; amino acid positions 582-598—helix; amino acid positions 600-602—beta strand; and amino acid positions 607-609—beta strand. The variants of ACE2 disclosed herein, including fragments of ACE2, may have or lack one or more of these structural features of ACE2.

ACE2 and the variants disclosed herein may include one or more of the following amino acid modifications: amino acid position 53—N-linked glycosylation; amino acid position 90—N-linked glycosylation; amino acid position 103—N-linked glycosylation; amino acid positions 133←→141—disulfide bond; amino acid position 322—N-linked glycosylation; amino acid positions 344←→361—disulfide bond; amino acid position 432—N-linked glycosylation; amino acid positions 530←→542; amino acid position 546—N-linked glycosylation; and amino acid position 690—N-linked glycosylation. The variants of ACE2 disclosed herein, including fragments of ACE2, may have or lack one or more of these amino acid modifications of ACE2 and/or may lack the amino acids thusly modified.

ACE2 regulates biological processes that may include: angiotensin catabolism processes in blood, angiotensin maturation processes, angiotensin-mediated drinking behavior processes, positive regulation of cardiac muscle contraction processes, positive regulation of gap junction assembly processes, positive regulation of reactive oxygen species metabolism processes, receptor biosynthesis processes, receptor-mediated virion attachment processes (e.g., coronaviruses), regulation of cardiac conduction processes, regulation of cell proliferation processes, regulation of cytokine production processes, regulation of inflammatory response processes, regulation of systemic arterial blood pressure by renin-angiotensin processes, regulation of vasoconstriction processes, regulation of vasodilation processes, tryptophan transport processes, and viral entry into host cell processes (e.g., coronaviruses). The variants of ACE2 disclosed herein, including fragments of ACE2, may regulate or may fail to regulate one or more of these biological processes.

The disclosed ACE2 variants may be modified so as to comprise an amino acid sequence, or modified amino acids, or non-naturally occurring amino acids, such that the disclosed ACE2 variants cannot be said to be naturally occurring. In some embodiments, the disclosed ACE2 variants are modified and the modification is selected from the group consisting of acylation, acetylation, formylation, lipolylation, myristoylation, palmitoylation, alkylation, isoprenylation, prenylation, and amidation. An amino acid in the disclosed polypeptides may be thusly modified, but in particular, the modifications may be present at the N-terminus and/or C-terminus of the polypeptides (e.g., N-terminal acylation or acetylation, and/or C-terminal amidation). The modifications may enhance the stability of the polypeptides and/or make the polypeptides resistant to proteolysis.

The disclosed ACE2 variants may include a non-naturally occurring N-terminal and/or C-terminal modification. For example, the N-terminal of the disclosed peptides may be modified to include an N-acylation or a N-pyroglutamate modification (e.g., as a blocking modification). The C-terminal end of the disclosed peptides may be modified to include a C-amidation. The disclosed peptides may be conjugated to carbohydrate chains (e.g., via glycosylation to glucose, xylose, hexose), for example, to increase plasma stability (notably, resistance towards exopeptidases).

The variants of ACE2 disclosed herein may be further modified. For example, the polypeptide fragment of ACE2 may be further modified to increase half-life in plasma and/or to enhance delivery to a target (e.g., the kidney, the lungs, the heart, etc.). In some embodiments, the polypeptide fragment is covalently attached to a polyethylene glycol polymer. In other embodiments, the polypeptide fragment may be conjugated to a nanoparticle (e.g., a biogel nanoparticle, a polymer-coated nanobin nanoparticle, and gold nanoparticles). Preferably, the polypeptide fragment of the disclosed methods of treatment and pharmaceutical compositions has a half-live in plasma of at least 6 hours, 12 hours, 1 day, 2 days, 3 days, 4 days, 5 days, 6 days, one week, two week, three weeks, four weeks, or longer. Strategies to improve plasma half-life of peptide and protein drugs are known in the art. (See Werle et al., “Strategies to improve plasma half life time of peptide and protein drugs,” Amino Acids 2006 Jun;30(4):351-67, the content of which is incorporated herein by reference in its entirety).

Pharmaceutical Compositions

The compositions disclosed herein may include pharmaceutical compositions comprising the presently disclosed bacterial toxins and formulated for administration to a subject in need thereof. Such compositions can be formulated and/or administered in dosages and by techniques well known to those skilled in the medical arts taking into consideration such factors as the age, sex, weight, and condition of the particular patient, and the route of administration.

The compositions may include pharmaceutical solutions comprising carriers, diluents, excipients, and surfactants, as known in the art. Further, the compositions may include preservatives (e.g., anti-microbial or anti-bacterial agents such as benzalkonium chloride). The compositions also may include buffering agents (e.g., in order to maintain the pH of the composition between 6.5 and 7.5).

The pharmaceutical compositions may be administered therapeutically. In therapeutic applications, the compositions are administered to a patient in an amount sufficient to elicit a therapeutic effect (e.g., a response which cures or at least partially arrests or slows symptoms and/or complications of disease (i.e., a “therapeutically effective dose”)).

### Illustrative Embodiments

The following embodiments are illustrative and should not be interpreted to limit the scope of the claimed subject matter.

Embodiment 1. A fusion protein comprising: (i) at least a portion of the ectodomain of human angiotensin converting enzyme 2 (ACE2) fused directly or via a linking sequence to (ii) at least a portion of the constant region of a human antibody; wherein the fusion protein binds to the spike protein of sudden acute respiratory syndrome coronavirus 2 (SARS-CoV-2) and the fusion protein comprises one or more mutations in ACE2 that result in reduced peptidase activity.

Embodiment 2. The fusion protein of embodiment 1, wherein the one or more mutations are selected from a mutation at position E145, R273, H345, P346, D368, H374, H378, E402, H505, or combinations thereof.

Embodiment 3. The fusion protein of embodiment 1 or 2, wherein the one or more mutations comprise a mutation at position R273.

Embodiment 4. The fusion protein of any of the foregoing embodiments, wherein the one or more mutations comprise the mutation R273A.

Embodiment 5. The fusion protein of any of the foregoing embodiments, wherein the one or more mutations comprise a mutation at position H378.

Embodiment 6. The fusion protein of any of the foregoing embodiments, wherein the one or more mutations comprise the mutation H378A.

Embodiment 7. The fusion protein of any of the foregoing embodiments, wherein the one or more mutations comprise a mutation at position E402.

Embodiment 8. The fusion protein of any of the foregoing embodiments, wherein the one or more mutations comprise the mutation E402A.

Embodiment 9. The fusion protein of any of the foregoing embodiments, wherein the fusion protein exhibits reduced peptidase activity for Angiotensin II of at least 50%, 60%, 70%, 80%, 90%, 95%, or 99% relative to a fusion protein that does not comprise the one or more mutations in ACE2.

Embodiment 10. The fusion protein of any of the foregoing embodiments, wherein the recombinant protein binds to sudden acute respiratory virus coronavirus 2 (SARS-CoV-2), for example, with an equilibrium dissociation constant (K_{d} (M)) of less than about 10⁻⁵, 10⁻⁶, 10⁻⁷, 10⁻⁸, 10⁻⁹, 10⁻¹⁰, 10⁻¹¹, or 10⁻¹².

Embodiment 11. The fusion protein of any of the foregoing embodiments, wherein the fusion protein inhibits binding to the spike protein of SARS-CoV-2 and has an EC₅₀ of no more than about 50 ng/ml, 40 ng/ml, 30 ng/ml, 25 ng/ml, 20 ng/ml, 15 ng/ml, or 10 ng/ml.

Embodiment 12. The fusion protein of any of the foregoing embodiments, wherein the fusion protein inhibits transduction of SARS-CoV-2 into cells that express ACE2 and has an IC₅₀ of no more than about 5 μg/ml, 2 μg/ml, 1 μg/ml, 0.5 μg/ml, 0.4 μg/ml, 0.3 μg/ml, or 0.2 μg/ml.

Embodiment 13. The fusion protein of any of the foregoing embodiments, wherein the fusion protein has a half-life 4(1/20 in blood of at least about 40 hours, 45 hours, 50 hours, or 55 hours.

Embodiment 14. The fusion protein of any of the foregoing embodiments, wherein the portion of the ectodomain of human angiotensin converting enzyme 2 (ACE2) comprises a sequence from amino acid S19 to amino acid F555 of SEQ ID NO:1 and further includes the one or more mutations in ACE2 that result in reduced peptidase activity.

Embodiment 15. The fusion protein of any of the foregoing embodiments, wherein the portion of the ectodomain of human angiotensin converting enzyme 2 (ACE2) comprises a sequence from amino acid S19 to amino acid K619 of SEQ ID NO:1 and further includes the one or more mutations in ACE2 that result in reduced peptidase activity.

Embodiment 16. The fusion protein of any of the foregoing embodiments, wherein the portion of the ectodomain of human angiotensin converting enzyme 2 (ACE2) comprises a sequence from amino acid S19 to amino acid S740 of SEQ ID NO:1 and further includes the one or more mutations in ACE2 that result in reduced peptidase activity.

Embodiment 17. The fusion protein of any of the foregoing embodiments, wherein the portion of the ectodomain of human angiotensin converting enzyme 2 (ACE2) does not comprise and is lacking the amino acid sequence 741-805 of SEQ ID NO:1.

Embodiment 18. The fusion protein of any of the foregoing embodiments, wherein the portion of the ectodomain of human angiotensin converting enzyme 2 (ACE2) comprises SEQ ID NO:5, SEQ ID NO:6, SEQ ID NO:7, SEQ ID NO:8, SEQ ID NO:9, SEQ ID NO:10, SEQ ID NO:11, SEQ ID NO:12, or SEQ ID NO:13.

Embodiment 19. The fusion protein of any of the foregoing embodiments, wherein the portion of the constant region of the human antibody is portion of a constant region of IgG1.

Embodiment 20. The fusion protein of any of the foregoing embodiments, wherein the portion of the constant region of the human antibody comprises the hinge region, CH2 region, and CH3 region.

Embodiment 21. The fusion protein of any of the foregoing embodiments, wherein the portion of the constant region of the human antibody comprises SEQ ID NO:14.

Embodiment 22. The fusion protein of any of the foregoing embodiments, wherein the fusion protein forms a dimer.

Embodiment 23. The fusion protein of any of the foregoing embodiments, wherein the fusion protein dimerizes via an intermolecular cysteine-cysteine disulfide bond formed between the portion of the constant region of the human antibody of one fusion protein and the portion of the constant region of the human antibody of another fusion protein.

Embodiment 24. The fusion protein of any of the foregoing embodiments, wherein the fusion protein comprises SEQ ID NO:15, SEQ ID NO:16, SEQ ID NO:17, SEQ ID NO:18, SEQ ID NO:19, SEQ ID NO:20, SEQ ID NO:21, SEQ ID NO:22, or SEQ ID NO:23.

Embodiment 25. The fusion protein of any of the foregoing embodiments, wherein the fusion protein comprises a linking sequence comprising 5-15 amino acids selected from glycine, serine, and alanine.

Embodiment 26. A pharmaceutical composition comprising the fusion protein of any of the foregoing embodiments and a suitable pharmaceutical carrier.

Embodiment 27. A method for treating and preventing infection by SARS-CoV-2 in a subject in need thereof, the method comprising administering to the subject the pharmaceutical composition of claim 26.

Embodiment 28. A polynucleotide encoding the fusion protein of any of embodiments 1-25.

Embodiment 29. A human angiotensin converting enzyme 2 (ACE2) protein comprising one or more mutations selected from a mutation at position E145, R273, H345, P346, D368, H374, H378, E402, H505, or combinations thereof.

Embodiment 30. The human ACE2 of embodiment 29, wherein the mutations are selected from E145A, R273A, H345A, P346A, D368A, H374A, H378A, E402A, H505A, or combinations thereof.

Embodiment 31. The human ACE2 of embodiment 29 or 30, comprising mutations at position R273, H378, E402, or combinations thereof.

Embodiment 32. The human ACE2 of any of claims 29-31, comprising mutations at position R273A, H378A, E402A, or combinations thereof.

Embodiment 33. The human ACE2 of any of claims 29-32, wherein the human ACE2 is soluble (e.g., wherein the human ACE2 does not comprise any portion of the transmembrane domain of ACE2 and/or the human ACE2 does not comprise any portion of the cytoplasmic domain of ACE2).

Embodiment 34. The human ACE2 of any of claims 29-33, wherein the human ACE2 binds to the spike protein of SARS-CoV-2.

Embodiment 35. A pharmaceutical composition comprising the human ACE2 of any of claims 29-34 and a suitable pharmaceutical carrier.

Embodiment 36. A method for treating and preventing infection by SARS-CoV-2 in a subject in need thereof, the method comprising administering to the subject the pharmaceutical composition of embodiment 35.

Embodiment 37. A polynucleotide encoding the human ACE2 of any of embodiments 29-34.

### Examples

The following examples are illustrative and should not be interpreted to limit the scope of the claimed subject matter.

### Example 1—ACE2-Fc Fusion Proteins Comprising Mutant ACE2 Sequences

ACE2 is the transmembrane human receptor for SARS-CoV-2. Human recombinant ACE2 (rhACE2) is being tested for its antiviral activity in competitive binding of the viral spike protein and therefore blocks viral entry of human cells. Previously, for an unrelated purpose we constructed a fusion between the ectodomain of ACE2 (aa: 1-740) and Fc of human IgG1. We showed the Fc-fusion strategy significantly extended the in vivo half-life of ACE2. Therefore, this invention seeks the use of the long-acting ACE2-Fc, representing an improvement from rhACE2, for the treatment of SARS-CoV-2. Additionally, because the physiologic activity of ACE2 is related to blood pressure (BP) and fluid regulation, rhACE2-Fc in theory will have a dual function as a therapeutic agent: 1. Antiviral, and 2. BP lowering. We followed a mutagenesis strategy to identify the catalytic residues of ACE2 for its physiologic substrates such as angiotensin II and apelin-13. To this end, we constructed and tested a panel of rhACE2(mutant)-Fc proteins. Three mutants R273A, H378A and E402A of rhACE2-Fc can individually inactivate ACE2 and therefore decouple the antiviral function from its cardiovascular effects through angiotensin II, apelin-13 and other vasoactive substrates of ACE2.

Previously (NU2016-163), we constructed ACE2-Fc as an injectable drug for blood pressure control. We showed its long-acting potential through rapid proteolytic degradation of angiotensin II. For the reason that ACE2 is also the human receptor of SARS-CoV-2, and others have proposed using injectable ACE2 as a competitive inhibitor of the virus, we anticipate the long-acting form that we constructed is superior to untagged soluble ACE2 as antiviral. It is however also noted that underlying cardiovascular conditions have a negative impact on the severity of viral infection. We followed a mutagenesis strategy to inactivate ACE2 to remove any unwanted cardiovascular effects of ACE2-Fc. Through this work, three rhACE29(mutant)-Fc constructs were identified (see attached results in Appendix).

The disclosed ACE2-Fc proteins may be utilized as antivirals for the treatment of SARS-CoV-2. In particular, the disclosed rhACE29(mutant)-Fc variants are longer-lasting than rhACE2, and have reduced cardiovascular effects as compared to rhACE2(wild-type)-Fc.

### REFERENCES

1. “Inhibition of SARS-CoV-2 Infections in Engineered Human Tissues Using Clinical-Grade Soluble Human ACE2.” Monteil V, Kwon H, Prado P, Hagelkruys A, Wimmer RA, Stahl M, Leopoldi A, Garreta E, Hurtado Del Pozo C, Prosper F, Romero JP, Wirnsberger G, Zhang H, Slutsky AS, Conder R, Montserrat N, Mirazimi A, Penninger JM. Cell. 2020 May 14; 181(4):905-913.
2. “Neutralization of SARS-CoV-2 Spike Pseudotyped Virus by Recombinant ACE2-Ig,” Changhai Lei 1, Kewen Qian 1, Tian Li 1, Sheng Zhang, Wenyan Fu, Min Ding 5, Shi Hu. Nat Commun. 2020 Apr. 24; 11(1):2070.

### Example 2—Designed Variants of ACE2-Fc that Decouple Anti-SARS-CoV-2 Activities from Unwanted Cardiovascular Effects

### Abstract

Angiotensin-converting enzyme 2 (ACE2) is the entry receptor for SARS-CoV-2, and recombinant ACE2 decoys are being evaluated as new antiviral therapies. We designed and tested an antibody-like ACE2-Fc fusion protein, which has the benefit of long pharmacological half-life and the potential to facilitate immune clearance of the virus. Out of a concern that the intrinsic catalytic activity of ACE2 may unintentionally alter the balance of its hormonal substrates and cause adverse cardiovascular effects in treatment, we performed a mutagenesis screening for inactivating the enzyme. Three mutants, R273A, H378A and E402A, completely lost their enzymatic activity for either surrogate or physiological substrates. All of them remained capable of binding SARS-CoV-2 and could suppress the transduction of a pseudotyped virus in cell culture. This study established new ACE2-Fc candidates as antiviral treatment for SARS-CoV-2 without potentially harmful side effects from ACE2's catalytic actions toward its vasoactive substrates.

### INTRODUCTION

As COVID-19 pandemic is still unfolding and no specific antiviral treatments are available, there is an unmet need to explore new drug candidates that are effective and safe, and broad spectrum against the evolving virus. In addition to the ongoing clinical trials of repurposed compounds and patient-derived antibodies, new drugs are being developed through targeted screening and rational design.

One of the focuses is on drug candidates that target receptor-mediated viral entry. A diverse group of human coronaviruses including SARS-CoV of 2002, HCoV-NL63 of 2004 and SARS-CoV-2 of COVID-19 rely on their spike proteins to bind angiotensin-converting enzyme 2 (ACE2) cell receptor as the first step in viral entry[1-4]. ACE2 is a membrane-bound carboxypeptidase that is best known for its activity against vasoactive peptides including angiotensin II, Apelin-13, among others[5]. These activities regulate cardiovascular functions and fluid balance. Additional functions of ACE2 were also discovered as chaperone of neutral amino acid transporter in intestine[6]. These enzymatic and nonenzymatic activities are not directly associated with ACE2's role as the gateway of viral entry. It has been shown that soluble ACE2 at generous abundance as compared to viral concentration can lower infectivity of cultured human cells, similar to experimental anti-spike antibodies[?-9]. Prior to the pandemic, one of the human recombinant soluble ACE2 (hrsACE2) drugs developed by Apeiron Biologics and GlaxoSmithKline (GSK) had completed Phase I and Phase II clinical trials for human pulmonary arterial hypertension and acute respiratory distress syndrome (ARDS) [10, 11], and is now repositioned for investigational treatment of COVID-19 (ClinicalTrials.gov identifier: NCTO4335136).

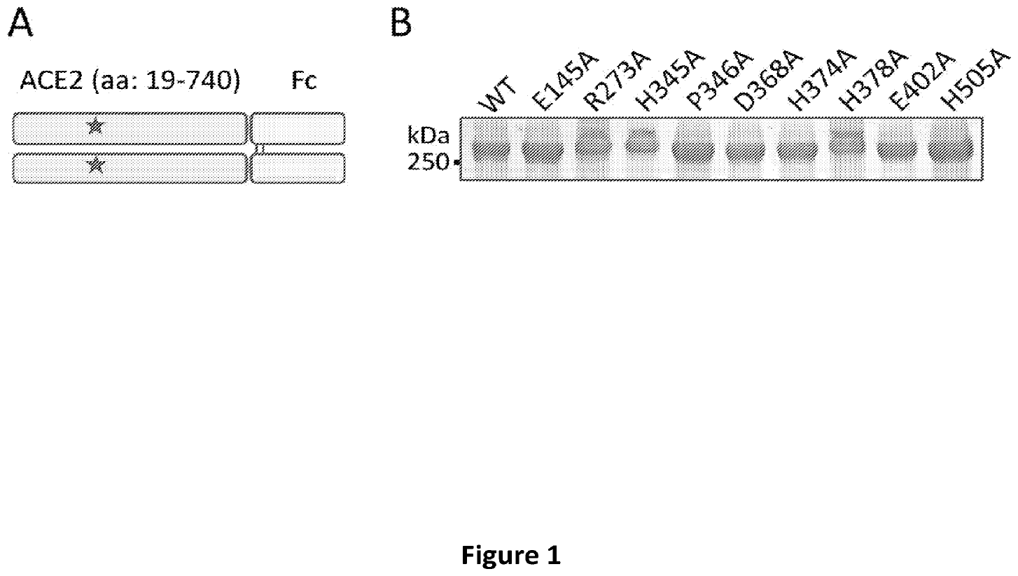

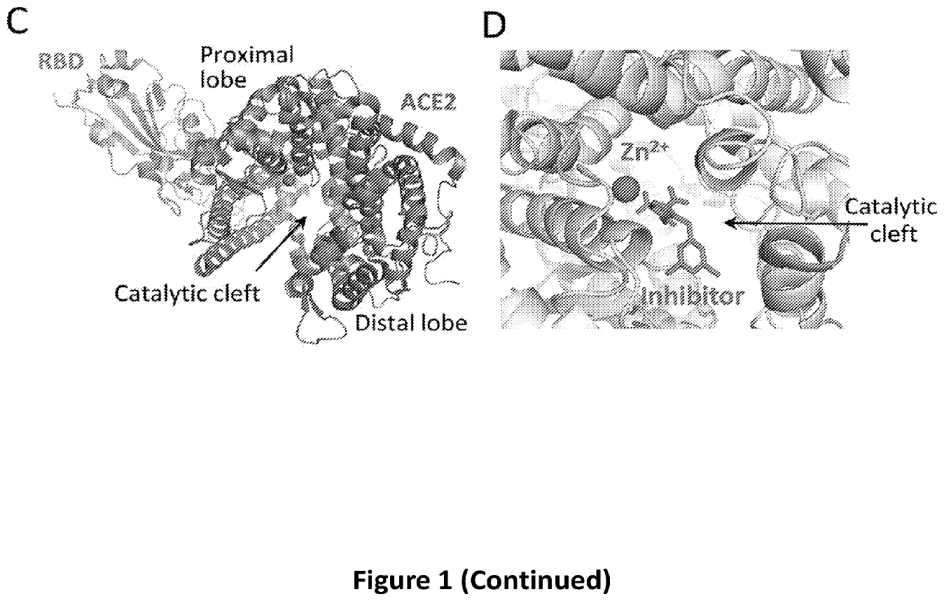

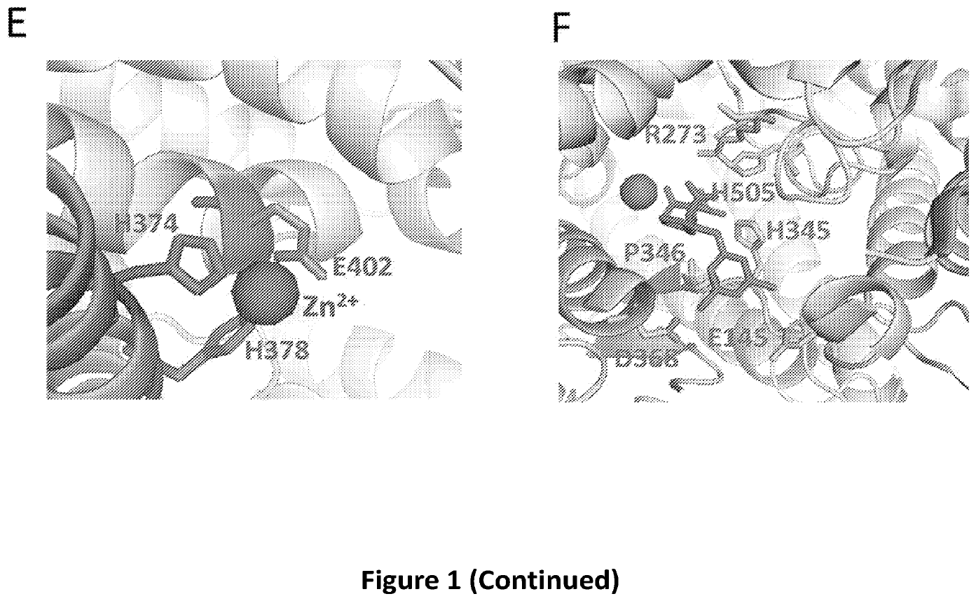

The current focus has been on improving binding affinity, pharmacokinetic/pharmacodynamic (PK/PD), antiviral specificity and neutralization efficacy of ACE2-based biologics through bioengineering design[8, 12-14]. Also in a different context unrelated to COVID-19, our group had previously constructed a chimeric fusion between the ectodomain of ACE2 and the Fc segment of IgG1 (“hinge” plus CH2 and CH3 regions) (FIG. 1A). In keeping with a well-recognized function of Fc to extend protein half-life through its cognate neonatal Fc receptor (FcRn), ACE2-Fc has improved pharmacokinetics as compared to untagged soluble ACE2[15]. The enzymatic activity of ACE2 in the fusion degraded angiotensin II (AngII) and rendered blood pressure control for up to two weeks. In comparison to its untagged counterpart to treat COVID-19, ACE2-Fc is predicted to offer superior pharmacological benefits, which make it also suitable for prophylactic usages by frontline healthcare workers and caregivers[13].

Our study attempts to address another potential drawback of hrsACE2 biologic. Although it was originally believed that the catalytic activity of ACE2 delivered in an excess quantity through therapeutic hrsACE2 may alleviate ARDS based on mouse studies[10, 16], the relevance in human disease remains unclear. COVID-19 mortality is prevalent among patients with underlying conditions such as cardiovascular disease, diabetes and chronic lung disease [17-20], a large number of COVID-19 patients are on existing angiotensin-converting-enzyme inhibitors (ACEI)/Angiotensin II receptor blockers (ARB) blockade therapy for preexisting cardiovascular and diabetic comorbidities[21]. There have been different opinions on whether Renin-Angiotensin-Aldosterone System (RAAS) blockade medications had improved or worsened COVID-19 recovery[22-26]. However, studies discovered a correlation between RAAS blockade and upregulation of endogenous ACE2 expression, causing concern of increased risk of SARS-CoV-2 infection[27-31]. The carboxypeptidase activity of therapeutic hrsACE2 hydrolyses a broad range of vasoactive hormonal substrates including AngII, apelin-13, bradykinin metabolites, among others, and exerts systemic RAAS blockade that affects the heart, the blood vessels, the kidney and the lung. Severe cases of SARS-CoV-2 infection frequently have multiorgan involvement [32-36]. In our view, the dual functions of investigational drug hrsACE2 to simultaneously act on viral neutralization and RAAS can potentially complicate clinical assessment of therapeutic efficacy. In order to achieve an exclusive antiviral function in an ACE2-derived biologic, we sought to modify ACE2 catalytic center to limit the catalysis of its vasoactive substrates.

Materials and Methods

Construction of ACE2-Fc mutant plasmids. DNA sequence encoding the ectodomain of human ACE2 (aa 1-740) was cloned from a human kidney cDNA library. DNA encoding human Fc of IgG1 has been described previously[15]. An in-frame fusion between ACE2 and Fc was constructed in pcDNA3 vector (Invitrogen, Carlsbad, Calif.). Site-directed mutagenesis by PCR was performed to create the panel of ACE2-Fc mutants. All mutants were confirmed by sequencing.

Recombinant protein expression and purification. The workflow for generating recombinant ACE2-Fc variants was similar to what had been reported before[15]. Briefly, HEK293 cells were transfected with individual ACE2-Fc variants by standard polyethylenimine (PEI) method. In transgene transfection studies, on day two of transfection cells were switched to serum-free DMEM. On day four, the culture media were harvested by centrifugation, and further concentrated using Amico Ultra Filters (Millipore, Billerica, Mass.). ACE2-Fc proteins were then purified by size-exclusion chromatography (SEC) using Superdex 200 Increase column (GE healthcare, Chicago, Ill.) and stored at −80° C. until used in experiments.

ΔACE2-Fc Arg273Ala, His378Ala and Glu402Ala, and wild-type ACE2-Fc selected for scaled production were produced from clonal stable-expressing cells. The general method was described previously[15]. Following plasmid transfection of HEK293 cells, the cells were selected under 1 mg/mL G418 (Thermo Fisher Scientific, Waltham, Mass.) for −14 days until isolated cell colonies appeared in the dishes. Individual cell clones were seeded into 96-well plates. When cells reached 50-100% density in the wells, the culture media were tested for their ACE2-Fc contents using a custom ELISA (anti-ACE2 antibody [Abcam, Cambridge, Mass.] for capturing and anti-human IgG-Fc-HRP [SouthernBiotech, Birmingham, Ala.] for detection). Clones with the highest expression of recombinant ACE2-Fc variants were selected, and individually expanded to five 150 mm dishes. Once reached ˜90% confluency, the cultures were switched to serum-free DMEM medium. After 4-5 days the culture media were harvested, and further concentrated using a VIVAFLOW 200 filtration system (100,000 MWCO by Sartorius, Stonehouse, UK). Recombinant ACE2-Fc proteins were purified using SEC as described above.

ACE2 peptidase activity measured using surrogate Mca-APK(Dnp). ACE2-Fc peptidase activity assay using surrogate fluorogenic substrate Mca-APK(Dnp) (Enzo Life Sciences, Farmingdale, N.Y.) was performed in black microtiter plates. The reaction buffer contained 50 mM 4-morpholineethanesulfonic acid, pH=6.5, 300 mM NaCl, 10 μM ZnC12, 0.01% Triton X-100 and 20 μM of Mca-APK(Dnp). The total reaction volume was 100 μL at room temperature and the duration of the reactions were 20 min. Peptidase activities were calculated as fluorescence intensity at 320 nm excitation and 420 nm emission wavelength.

ACE2 peptidase activity measured using physiological substrates of AngII and apelin-13. We described the method previously and referred to the workflow as Phenylalanine Assay, which was tested using AngII and apelin-13 as substrates in reactions with ACE2[37]. It involves two coupled reactions, hydrolysis of the C-terminus phenylalanine residues from the substrates by ACE2 catalysis and the measurement of free amino acid phenylalanine using yeast enzyme of phenylalanine ammonia lyase (PAL) in a colorimetric assay (supplementary Figure S2).

The reactions were carried out using indicated concentrations of ACE2-Fc proteins together with either AngII or apelin-13 at indicated concentrations in reaction buffer containing 20 mM Tris-HCl, pH=7.4, 136 mM NaCl and 10 μM ZnC12. The first reaction was proceeded at 37° C. after 20 min before stopped by 80° C. heat inactivation for 5 min. The second reaction used a phenylalanine detection kit (Sigma-Aldrich, St. Louis, Mo.). 1 μL enzyme mix and 1 μL developer from the kit were added to the above reaction, which was allowed to proceed for 20 min at room temperature. Fluorescence intensity was measured at 535 nm excitation and 585 nm emission wavelength, and all reactions were performed in triplicate.

SARS-CoV-2 RBD binding assay. Recombinant viral RBD protein was purchased from Sino Biological (Beijing, China). ELISA wells were precoated with either PBS as controls or 100 ng/well of RBD protein. Serial concentrations of individual ACE2-Fc variants were added to the wells. After overnight incubation at 4° C., the wells were washed three times with TBST buffer before HRP-conjugated anti-human IgG-Fc secondary antibody was added. HRP reactions were developed with TMB substrate and the binding strength derived from OD450 (nm) readings of the reactions. The EC50 values were determined by log(agonist) vs. response nonlinear regression fit analysis (GraphPad Prism).

Inhibition of viral transduction with purified AACE2-Fc mutants. Spike (SARS-CoV-2) pseudotyped lentivirus with luciferase reporter gene was purchased from BPS Bioscience (San Diego, Calif.). The virus was used to transduce HEK293 with stable expression of receptor full-length ACE2. The stable cell line was created from plasmid expressing full-length human ACE2 in pcDNA3 vector. Followed a similar procedure for generating ACE2-Fc clones, stable HEK293 clones with receptor ACE2 expression was identified using immune detection of ACE2 in the cells. The ACE2-HEK293 cells were seeded at a density of 10,000 cells per well into white 96-well cell culture microplate one day before transduction. To test inhibition of viral transduction, 5 μL pseudotyped lentivirus were preincubated with 5 μL vehicle or serially diluted ACE2-Fc variants at 37° C. for 1 h and then added into the cells. After overnight incubation, the cells were refed with fresh medium and incubated for another 36 hours. Luciferase activity was measured using ONE-Glo™ Luciferase Assay System according to the manufacture's protocol (Promega, Madison, Wis.). The IC₅₀ values were determined by log(inhibitor) vs. response nonlinear regression fit analysis (GraphPad Prism).

ACE2-Fc pharmacokinetics in mice. Institutional Animal Care and Use Committee of the Northwestern University approved the animal procedure in this study (approved protocol number IS00009990). The general method for pharmacokinetic measurements were described previously[15]. Briefly, a bolus intravenous injection of ACE2-Fc proteins (0.5 mg/kg body weight) was performed in 10 weeks old female BALB/c mice.

Subsequently, serial blood samples were collected from tail bleeding at indicated time points. Collected blood samples were left undisturbed on ice, and sera were isolated by centrifugation at 6000×g for 10 minutes at 4° C. The levels of ACE2-Fc in the sera were measured by ELISA using anti-ACE2 capturing antibody and anti-human IgG-Fc-HRP antibody for detection as described above.

Results

ACE2-Fc mutagenesis strategy to remove catalytic activity. We constructed an ACE2-Fc template by using the ectodomain of ACE2 fused with an Fc sequence (FIG. 1A). The chimeric fusion naturally formed a dimer of >250 kDa as expected (FIG. 1B). There is an extensive amount of information with regard to the structural characteristics of ACE2 in relationship to SARS-CoV-2 receptor-binding domain (RBD)[1, 2, 38-40]. SARS-CoV-2 binds a surface segment of ACE2 through the apex of its spike protein (FIG. 1C). ACE2 is a metallopeptidase that requires divalent cation such as zinc for activity. A Zn2+ion is buried deep in the catalytic cleft within the proximal lobe relative to the viral binding site. Based on an inhibitor-bound structure of ACE2[41], both proximal and distal residues that line the catalytic cleft form interactions with the inhibitor, which occupies the presumed substrate pocket (FIG. 1D). Zn²⁺ is coordinated by three residues, His374, His378 and Glu402, which are the obvious choices for mutagenesis when making enzymatically inactive mutants[8] (FIG. 1E). In addition to these Zn²⁺-binding sites, we sought to look for catalytic residues in contact with the substrates that are further away from RBD binding segment. The inhibitor-bound structure indicates six residues that extend their side chains toward the substrate direction. These are Glu145, Arg273, His345, Pro346, Asp368 and His505 (FIG. 1F).

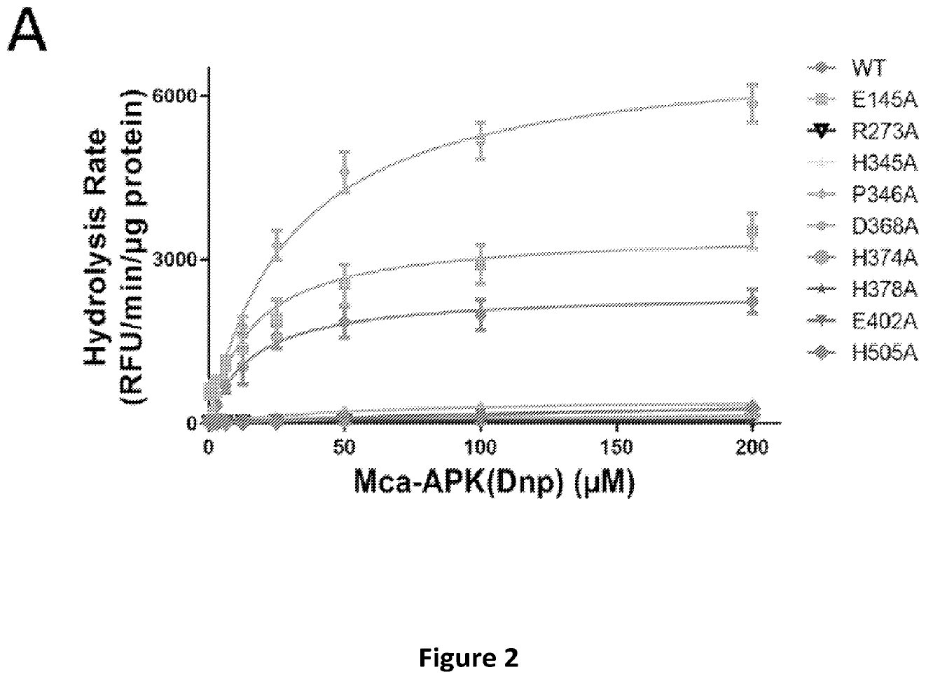

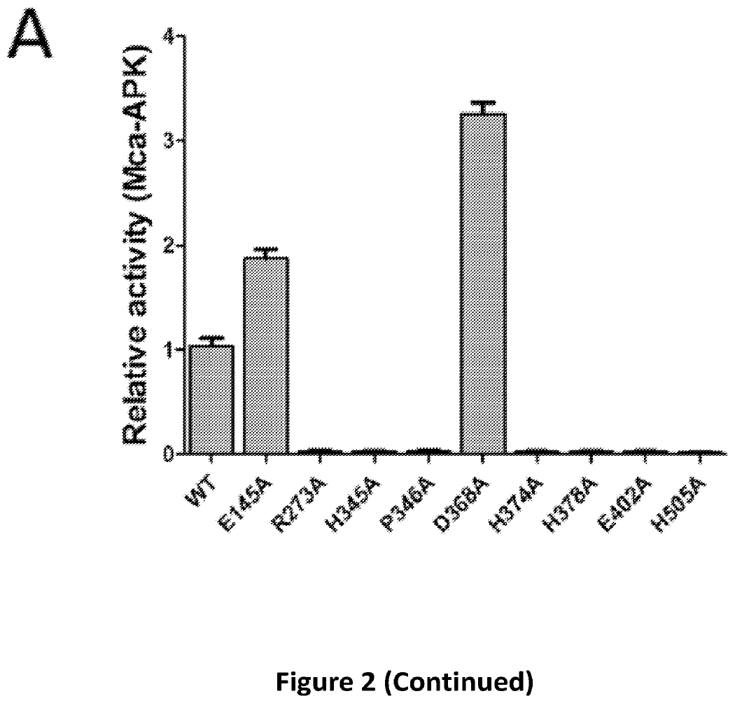

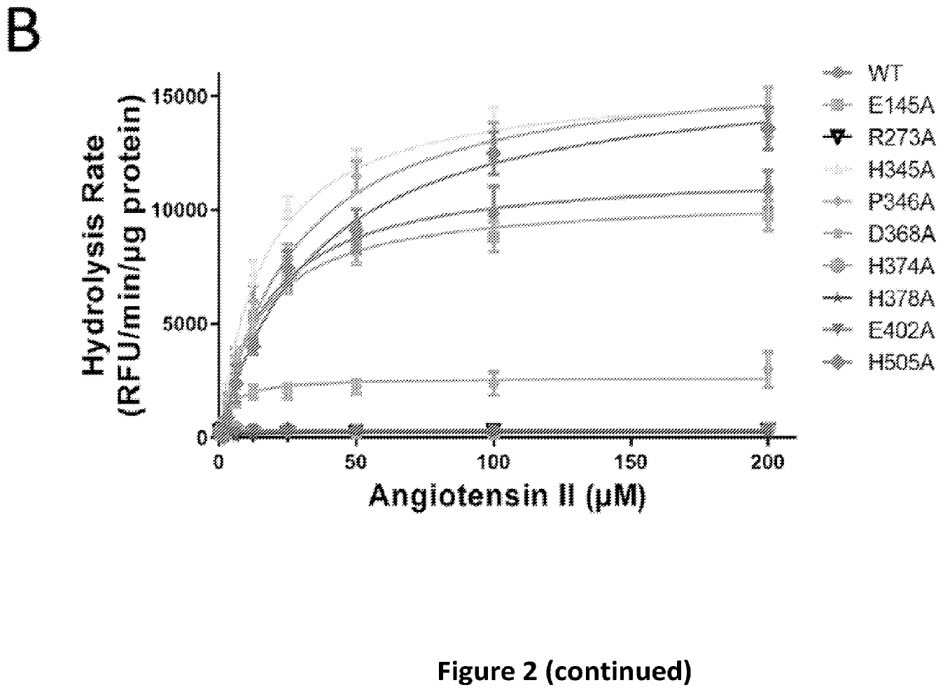

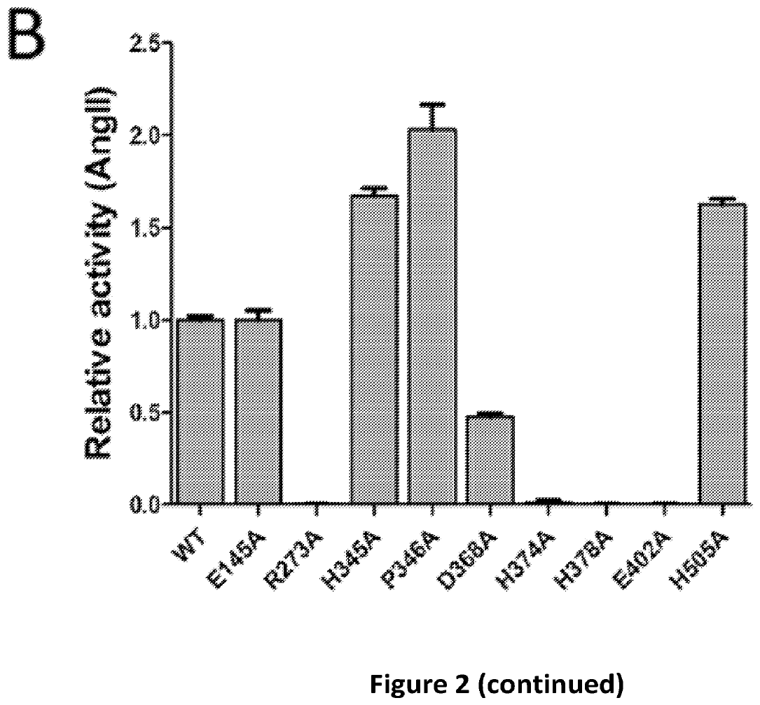

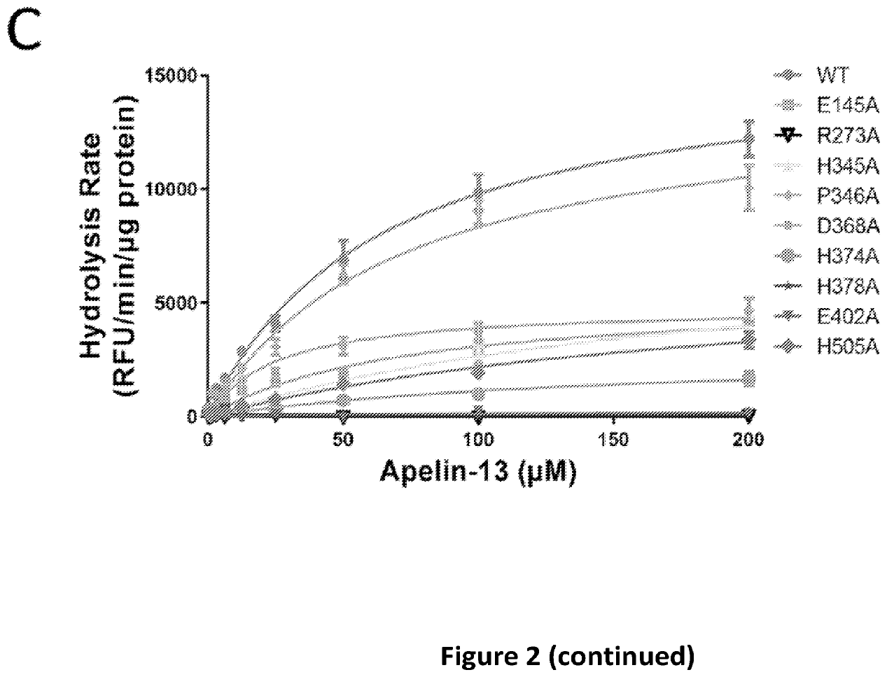

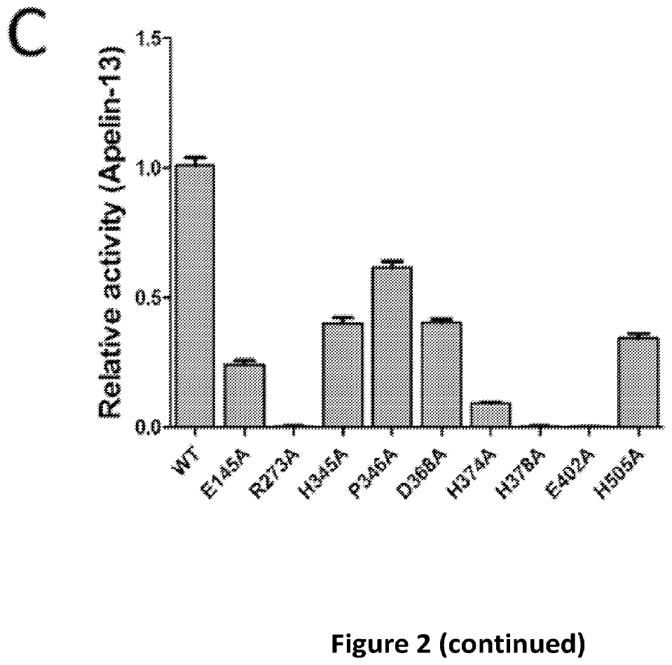

Substrate-dependent inactivation among ACE2-Fc mutants. Next, we made alanine-substitution of each residue including the three that bind Zn2+ and the additional six that potentially bind substrates (supplementary FIG. S1). Wild-type and 9 mutants of ACE2-Fc were produced using a HEK293 expression system as soluble proteins (FIG. 1B). Purified proteins were subjected to a set of enzymatic assays using a surrogate Mca-APK(Dnp) fluorogenic substrate and physiological substrates such as AngII and apelin-13 (FIG. 2). It should be noted that although the surrogate substrate Mca-APK(Dnp) is traditionally used for measuring ACE2 activity, the sequence does not resemble those of the physiological substrates, which share a Pro-Phe motif at their C-termini (Supplementary figure S2). Instead, the catalysis of AngII and apelin-13 by ACE2-Fc variants was measured by the hydrolysis rate of their C-terminus Phe amino acid[37].

We performed Mca-APK(Dnp) measurements, alongside phenylalanine hydrolysis assays using AngII and apelin-13 as substrates to determine the enzymatic activities of the ACE2-Fc variants (FIG. 2A-C). In order to better characterize the catalytic performances of individual ACE2-Fc mutants relative to their wild-type counterpart, we conducted two types of enzymatic assays. The first method used an excess amount of the enzyme (100 ng) in reactions with varying concentrations of the three substrates. This would potentially detect low partial activity of enzymes. The second method had a lower amount of purified ACE2-Fc variants (10 ng each) to react with an excess quantity of the substrates (2 or 10 nmol: see Methods) in order to distinguish among mutants with high activities. As it turned out, the results from these two methods were to an extend in agreement with each other (FIG. 2: left panels compared to right panels). One of the surprising findings was that there was a clear evidence of substrate-dependent inactivation of individual mutations, particularly among those lining the inhibitor/substrate space. For instance, while Mca-APK(Dnp) showed no activity of 7 ACE2-Fc mutants, including 4 substrate-binding residues of Arg273Ala, His345Ala, Pro346Ala and His505Ala (FIG. 2A), His345Ala, Pro346Ala and His505Ala remained active toward AngII and apelin-13 (FIG. 2B, 2C). These differences may attribute to unique steric conformation of the catalytic pocket for each substrate, and therefore individual ACE2 residues display distinct substrate-specific importance as seen in our results. In addition, His374Ala, one of the Zn²⁺-binding residues, retained a low level of activity against Apelin-13 (FIG. 2C). When all three substrates are considered, Arg273Ala, His378Ala and Glu402Ala were completely lack of peptidase activity. We referred these three mutants to as ΔACE2-Fc (Δ: loss-of-activity) and considered them as candidate variants emerged from the screen.

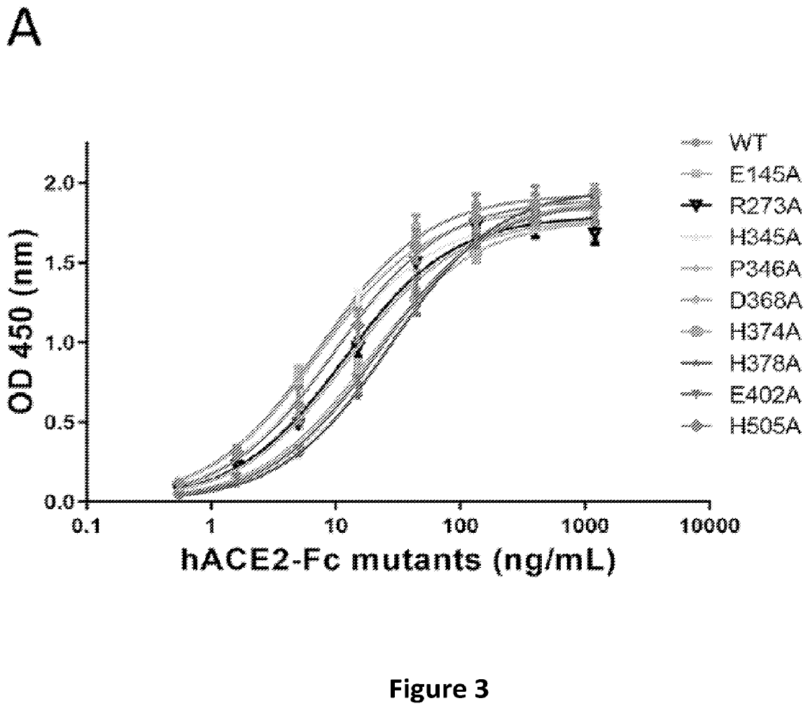

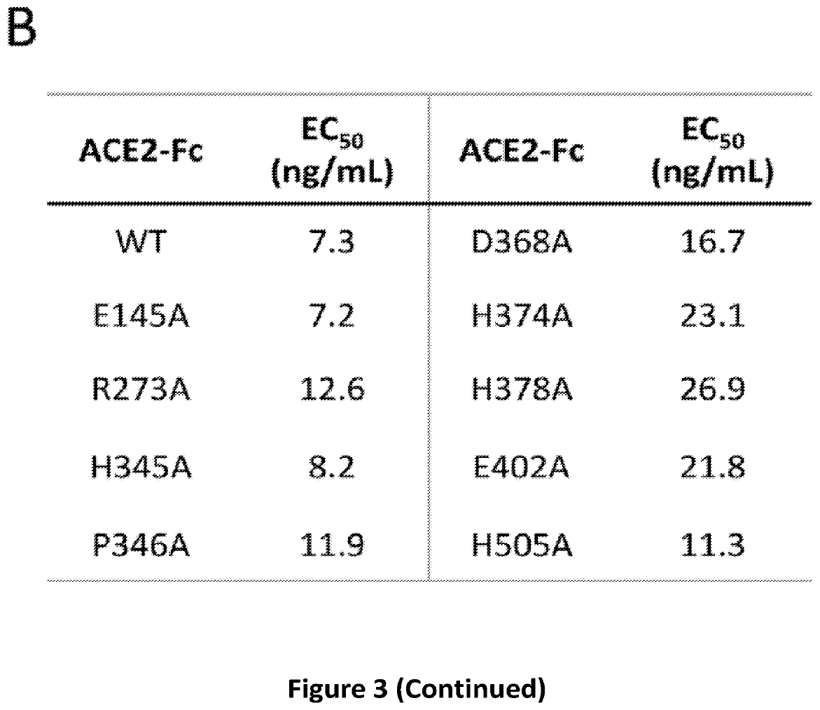

Binding affinities of individual ACE2-Fc mutants to SARS-CoV-2 receptor-binding domain. Next, we performed binding assays using individual variants against purified spike RBD protein. As expected, all mutants displayed similar levels of binding to viral RBD, considering the relatively minor changes from the point mutations made to ACE2-Fc (FIG. 3). Overall, the wild-type protein showed the highest binding affinity, whereas all three Zn²⁺-binding site mutants, His374Ala, His378Ala and Glu402Ala, had the lowest affinities to RBD. This is consistent with the expectation that the ion pocket is in proximity to the viral binding site on ACE2 (FIG. 1C), and also that changing ion-binding can potentially induce structurally instability of the protein. In contrast, Arg273Ala mutant of the substrate-binding pocket, which showed complete loss-of-activity towards all three substrates, is situated on the distal lobe and is less likely to affect desired viral binding.

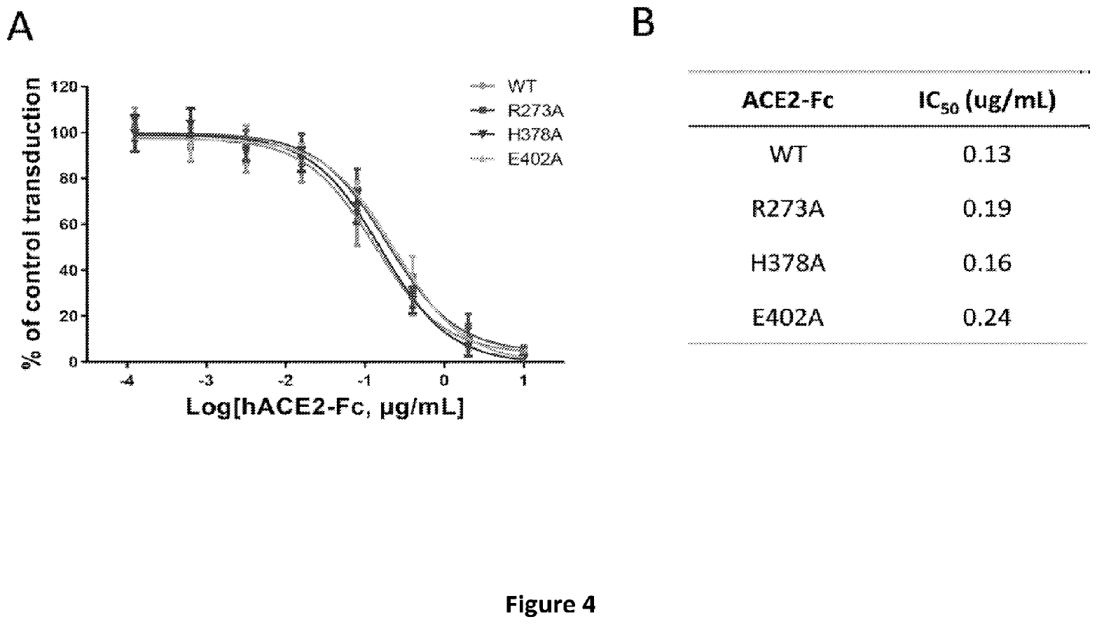

Competitive inhibition of pseudotyped viral transduction by R273A, H378A and E402A mutants of ΔACE2-Fc. ΔACE2-Fc Arg273Ala, His378Ale and Glu402Ala, and wild-type ACE2-Fc proteins were further tested for their antiviral potency. We conducted a series of viral inhibition assays using a pseudotyped reporter virus decorated with SARS-CoV-2 spike protein. The virus was able to transduce HEK293 cells that express full-length receptor ACE2 (See Methods). Each of the four ACE2-Fc variants in a range of concentrations was added to culture medium to test the potential of viral inhibition. As expected, all variants showed similar levels of efficacy to block pseudoviral transduction (FIG. 4A), with wild-type ACE2-Fc had a leading IC₅₀ of 0.13 μg/mL, followed by His378Ala, Arg273Ala and Glu402Ala with their IC₅₀s of 0.16 μg/mL, 0.19 μg/mL and 0.25 μg/mL, respectively (FIG. 4B). In terms of neutralization activity of ACE2-Fc, we expect the pseudotyped virus and authentic SARS-CoV-2 react to the biologic similarly.

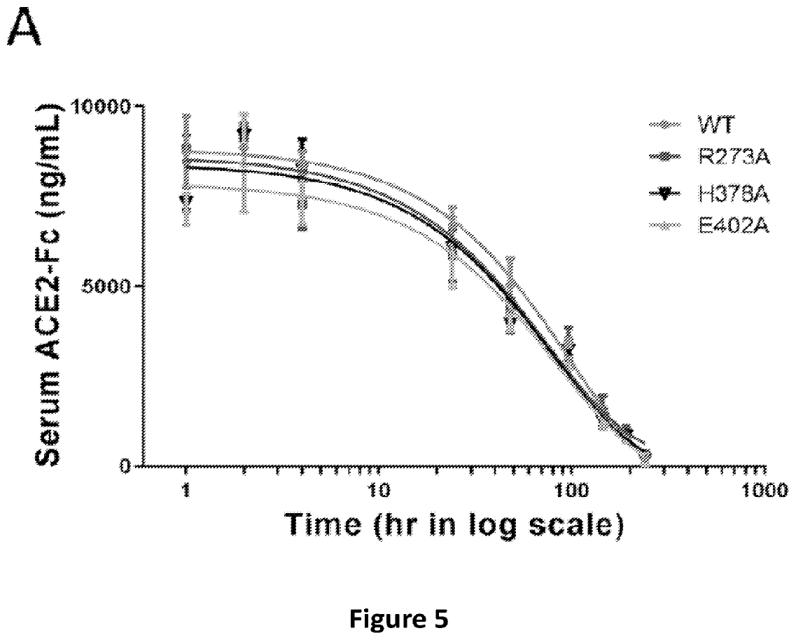

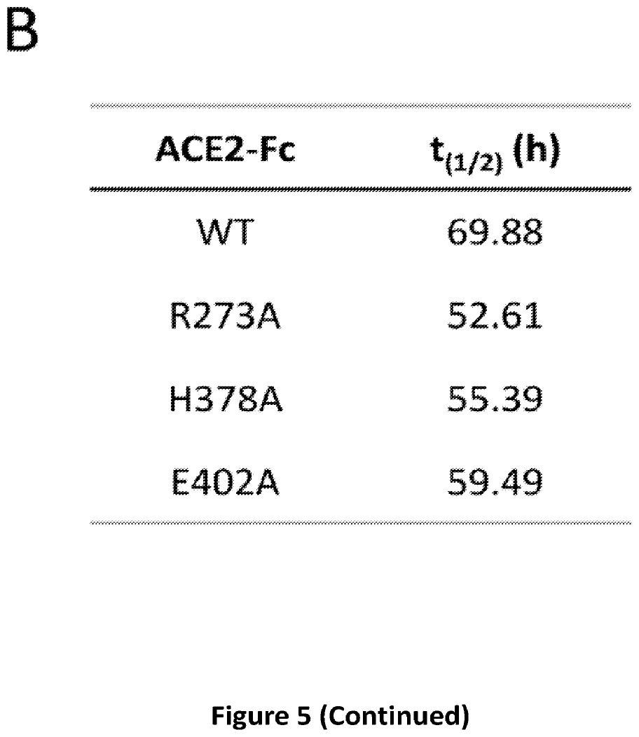

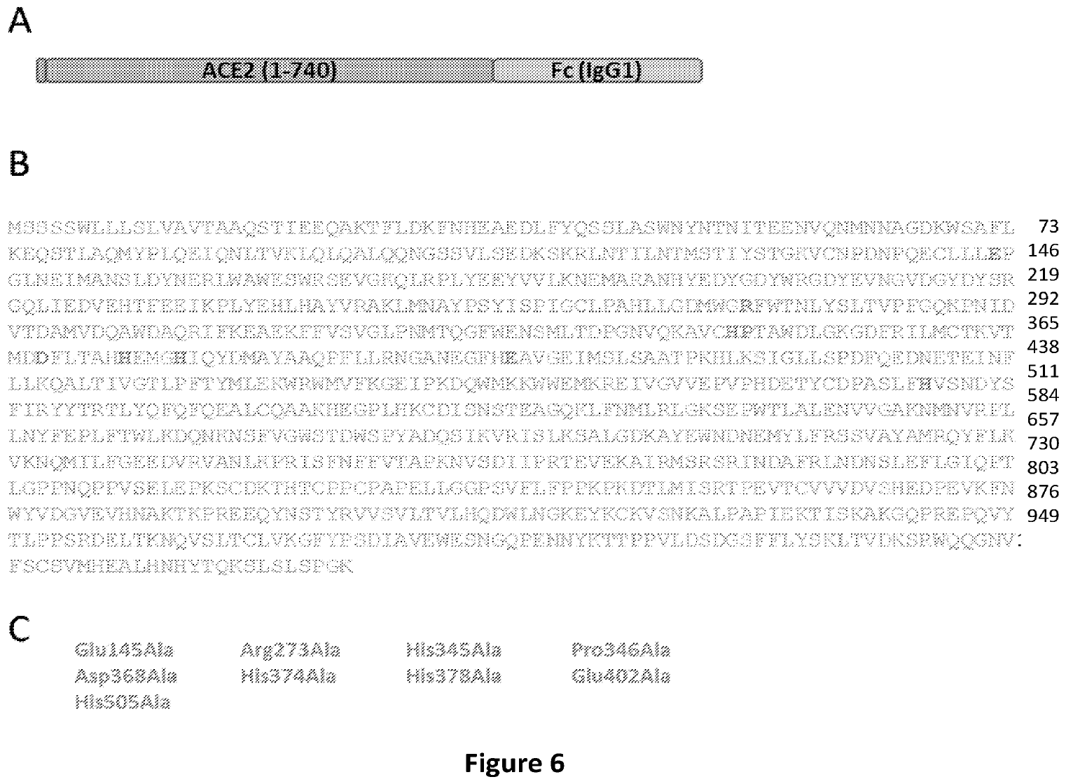

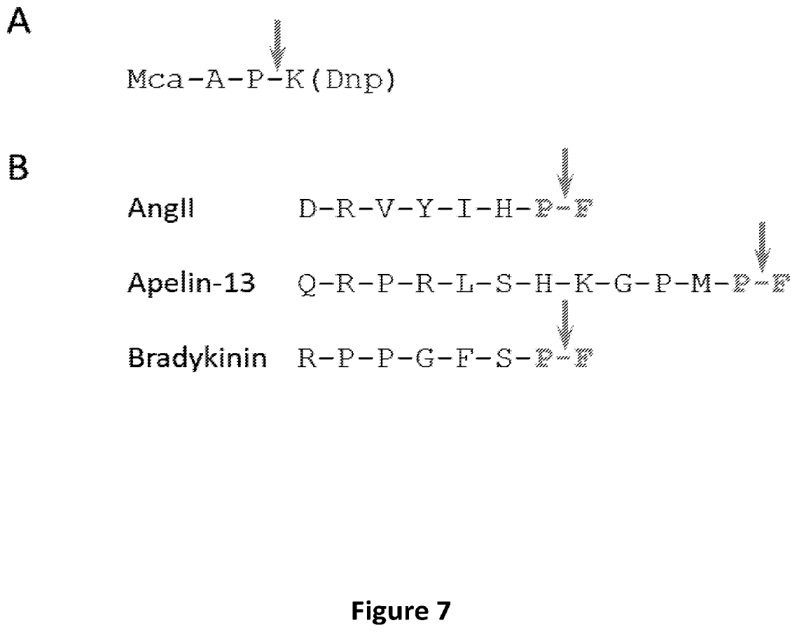

Pharmacokinetics of lead ΔACE2-Fc proteins. We have previously shown the in vivo longevity of mouse ACE2-Fc, as well as the fact that mouse FcRn recognizes human Fc[15]. Here we intravenously injected the ACE2-Fc variants in mice and measured pharmacokinetics of biologics. All three ΔACE2-Fc and their wild-type control exhibited long half-lives in the range between 52.61 hrs and 69.88 hrs (FIG. 5), consistent with the expectation for Fc-fusion proteins.

### DISCUSSION

Our study followed a design strategy of screening for ACE2-Fc variants of having an exclusive SARS-CoV-2 affinity with the absence of enzymatic activity towards vasoactive substrates. Based on the structure of ACE2's catalytic center, we selected a total of 9 residues to be individually replaced with alanine. These included 3 residues with their side chains binding to divalent cation and 6 residues that line the substrate pocket. We used a surrogate fluorogenic substrate as well as two physiological substrates of ACE2 in reactions and discovered an unexpected substrate-dependent inactivation among individual mutants of ACE2-Fc. The screening identified three loss-of-activity variants (ΔACE2-Fc), one with a mutation of substrate-binding site and two others having impairment of cation-binding. All three lead candidates maintained their binding capacity towards SARS-CoV-2 spike protein and inhibited the transduction of a pseudotyped reporter virus.

Although we focused on inactivating ACE2 enzymatic activity to separate its actions on RAAS from SARS-CoV-2 neutralization, it has been widely speculated that the dual actions may benefit treatment of COVID-19. As ACE2 catalyzes the conversion of AngII to Ang-(1-7), therapeutic hrsACE2 or ACE2-Fc will change the balance from AngII-mediated stimulation of AT1 receptor to AT2 and/or Mas receptor activation, which may reduce pulmonary dysfunction due to AT1 associated inflammatory responses, lung edema and ARDS[17, 42-45]. Since there is no clinical data on ACE2-derived antiviral therapies, the hypotheses about beneficial RAAS inhibition are based on observations of COVID-19 patients who are on existing ACEI or ARB treatments. The general consensus is that these patients should continue RAAS blockade therapies for treating comorbidities during recovering from viral infection.

One of the main benefits of ACE2-Fc fusion construction is its long-acting time as compared to recombinant ACE2 without the tag[15]. It is expected to provide important assurance of sufficient drug levels to counteract the fluctuating levels of virions in patients, particularly during viremia. Based on clinical knowledge of Fc-tagged Factor VIII (ELOCTATE®) used in hemophilia A patients, dosing at 3-5 day intervals is sufficient to maintain a high blood level of the drug (US FDA recommendation).

The structural arrangement of ACE2-Fc resembles that of an antibody, with the replacement of antigen-binding Fab portion of antibody with ACE2 to bind SARS-CoV-2 spike. Meanwhile, the Fc portion can potentially induce immunological clearance of the virus, which, together in a fusion with ACE2, may be an effective immunoadhesin[13, 46] to trigger complement activation, antibody-mediated cytotoxicity and opsonization, and agglutination of targets. With respect to the potential antibody-like benefits of ACE2-Fc, we compare our overall strategy with existing CD4 immunoadhesin (termed PRO 542) that has existing clinical data for the treatment of HIV infection[47-51]. PRO 542 (CD4-IgG2/Fc with CD4 targeting HIV gp120) antiviral is a tetravalent fusion protein using the constant region of IgG2 as opposed to IgG1 of ACE2-Fc. One notable difference is that IgG2 has extremely low affinity to FcγRs on phagocytic cells, while both IgG1 and IgG2 can activate complement. With regard to our proof-of-principle study of ACE2-Fc as candidate antiviral drugs, it is important to point out that in terms of choices of the Fc tag, there are alternative strategies in recombinant construction. Nevertheless, the inclusion of immunoadhesin potentials of the antiviral may have caveats. While it may certainly boost immune clearance of the virus, such as through FcγR's selective binding of clustered Fc, it may also elevate complement and cytokine responses to further aggravate the inflammation. Although these adverse side effects can be mitigated through modifications of the Fc domain, the ultimate therapeutic effects in the context of individual patients' conditions can only be determined through rigorous clinical studies.

With regard to drug toxicity, our mouse study of repeated doses of ACE2-Fc in mice had showed the biologic to be well tolerated for up to two months[15]. However, we cannot extrapolate that its will also be safe COVID-19 patients. As we consider it is not a simple neutralizing agent of the virus, its bifurcated ACE2 head groups can possibly trigger agglutination of the virus that can potentially aggravate the hypercoagulable state, making the drug less tolerable in these conditions.

From the perspective of recombinant manufacturing, there are challenges ahead for the simple fact that ACE2-Fc is a large protein (˜130 kDa as a monomer and −260 kDa as a dimer). Furthermore, cocrystal structures of ACE2 with an inhibitor showed large movements of the two lobes as compared to the Apo structures[41], suggesting an intrinsic instability of ACE2 protein. Also of note is an earlier study by Lei et al using double mutations of His374 and His378 of the zinc-binding pocket for neutralization of SARS-CoV-2[8]. However, the ion is important in maintaining protein structure and stability of metallopeptidases[52] [53, 54]. This double mutation, as well as our His378Ala and Glu402Ala single mutations of the zinc-binding pocket may potentially suffer protein instability problems. In addition, multi-site mutagenesis increases the risk of structural instability and antigenic potentials. On the other hand, the Arg273Ala single site mutant that is predicted to change the substrate pocket will likely have a milder impact on overall protein stability, which is an important parameter in pharmaceutical production.

### REFERENCES

[1] R. Yan, Y. Zhang, Y. Li, L. Xia, Y. Guo, Q. Zhou, Structural basis for the recognition of SARS-CoV-2 by full-length human ACE2, Science 367(6485) (2020) 1444-1448.
[2] D. Wrapp, N. Wang, K. S. Corbett, J. A. Goldsmith, C. L. Hsieh, 0. Abiona, B. S. Graham, J. S. McLellan, Cryo-EM structure of the 2019-nCoV spike in the prefusion conformation, Science 367(6483) (2020) 1260-1263.
[3] P. Zhou, X. L. Yang, X. G. Wang, B. Hu, L. Zhang, W. Zhang, H. R. Si, Y. Zhu, B. Li, C. L. Huang, H. D. Chen, J. Chen, Y. Luo, H. Guo, R. D. Jiang, M. Q. Liu, Y. Chen, X. R. Shen, X. Wang, X. S. Zheng, K. Zhao, Q. J. Chen, F. Deng, L. L. Liu, B. Yan, F. X. Zhan, Y. Y. Wang, G. F. Xiao, Z. L. Shi, A pneumonia outbreak associated with a new coronavirus of probable bat origin, Nature 579(7798) (2020) 270-273.
[4] M. Hoffmann, H. Kleine-Weber, S. Schroeder, N. Kruger, T. Herrler, S. Erichsen, T. S. Schiergens, G. Herrler, N. H. Wu, A. Nitsche, M. A. Muller, C. Drosten, S. Pohlmann, SARS-CoV-2 Cell Entry Depends on ACE2 and TMPRSS2 and Is Blocked by a Clinically Proven Protease Inhibitor, Cell 181(2) (2020) 271-280 e8.
[5] C. Vickers, P. Hales, V. Kaushik, L. Dick, J. Gavin, J. Tang, K. Godbout, T. Parsons, E. Baronas, F. Hsieh, S. Acton, M. Patane, A. Nichols, P. Tummino, Hydrolysis of Biological Peptides by Human Angiotensin-converting Enzyme-related Carboxypeptidase, 277(17) (2002) 14838-14843.
[6] S. M. R. Camargo, D. Singer, V. Makrides, K. Huggel, K. M. Pos, C. A. Wagner, K. Kuba, U. Danilczyk, F. Skovby, R. Kleta, J. M. Penninger, F. Verrey, Tissue-Specific Amino Acid Transporter Partners ACE2 and Collectrin Differentially Interact With Hartnup Mutations, Gastroenterology 136(3) (2009) 872-882.e3.
[7] V. Monteil, H. Kwon, P. Prado, A. Hagelkruys, R. A. Wimmer, M. Stahl, A. Leopoldi, E. Garreta, C. Hurtado Del Pozo, F. Prosper, J. P. Romero, G. Wirnsberger, H. Zhang, A. S. Slutsky, R. Conder, N. Montserrat, A. Mirazimi, J. M. Penninger, Inhibition of SARS-CoV-2 Infections in Engineered Human Tissues Using Clinical-Grade Soluble Human ACE2, Cell 181(4) (2020) 905-913 e7.
[8] C. Lei, K. Qian, T. Li, S. Zhang, W. Fu, M. Ding, S. Hu, Neutralization of SARS-CoV-2 spike pseudotyped virus by recombinant ACE2-Ig, Nat Commun 11(1) (2020) 2070.
[9] N. Iwanaga, L. Cooper, L. Rong, B. Beddingfield, J. Crabtree, R. A. Tripp, J. K. Kolls, Novel ACE2-IgG1 fusions with improved activity against SARS-CoV2, bioRxiv (2020).
[10] A. Khan, C. Benthin, B. Zeno, T. E. Albertson, J. Boyd, J. D. Christie, R. Hall, G. Poirier, J. J. Ronco, M. Tidswell, K. Hardes, W. M. Powley, T. J. Wright, S. K. Siederer, D. A. Fairman, D. A. Lipson, A. I. Bayliffe, A. L. Lazaar, A pilot clinical trial of recombinant human angiotensin-converting enzyme 2 in acute respiratory distress syndrome, Crit Care 21(1) (2017) 234.
[11] A. R. Hemnes, A. Rathinasabapathy, E. A. Austin, E. L. Brittain, E. J. Carrier, X. Chen, J. P. Fessel, C. D. Fike, P. Fong, N. Fortune, R. E. Gerszten, J. A. Johnson, M. Kaplowitz, J. H. Newman, R. Piana, M. E. Pugh, T. W. Rice, I. M. Robbins, L. Wheeler, C. Yu, J. E. Loyd, J. West, A potential therapeutic role for angiotensin-converting enzyme 2 in human pulmonary arterial hypertension, Eur Respir J 51(6) (2018).
[12] K. K. Chan, D. Dorosky, P. Sharma, S. A. Abbasi, J. M. Dye, D. M. Kranz, A. S. Herbert, E. Procko, Engineering human ACE2 to optimize binding to the spike protein of SARS coronavirus 2, Science (2020).
[13] R. L. Kruse, Therapeutic strategies in an outbreak scenario to treat the novel coronavirus originating in Wuhan, China, F1000Res 9 (2020) 72.
[14] X. Miao, Y. Luo, X. Huang, S. M. Y. Lee, Z. Yuan, Y. Tang, L. Chen, C. Wang, F. Wu, Y. Xu, W. Jiang, W. Gao, X. Song, Y. Yan, T. Pang, C. Chen, Y. Zou, W. Fu, L. Wan, J. Gilbert-Jaramillo, M. Knight, T. K. Tan, P. Rijal, A. Townsend, J. Sun, X. Liu, W. James, A. Tsun, Y. Xu, A novel biparatopic hybrid antibody-ACE2 fusion that blocks SARS-CoV-2 infection: implications for therapy, MAbs 12(1) (2020) 1804241.
[15] P. Liu, J. Wysocki, T. Souma, M. Ye, V. Ramirez, B. Zhou, L. D. Wilsbacher, S. E. Quaggin, D. Batlle, J. Jin, Novel ACE2-Fc chimeric fusion provides long-lasting hypertension control and organ protection in mouse models of systemic renin angiotensin system activation, Kidney Int 94(1) (2018) 114-125.
[16] Y. Imai, K. Kuba, S. Rao, Y. Huan, F. Guo, B. Guan, P. Yang, R. Sarao, T. Wada, H. Leong-Poi, M. A. Crackower, A. Fukamizu, C. C. Hui, L. Hein, S. Uhlig, A. S. Slutsky, C. Jiang, J. M. Penninger, Angiotensin-converting enzyme 2 protects from severe acute lung failure, Nature 436(7047) (2005) 112-6.
[17] G. P. Rossi, V. Sanga, M. Barton, Potential harmful effects of discontinuing ACE-inhibitors and ARBs in COVID-19 patients, Elife 9 (2020).
[18] T. J. Guzik, S. A. Mohiddin, A. Dimarco, V. Patel, K. Savvatis, F. M. Marelli-Berg, M. S. Madhur, M. Tomaszewski, P. Maffia, F. D′Acquisto, S. A. Nicklin, A. J. Marian, R. Nosalski, E. C. Murray, B. Guzik, C. Berry, R. M. Touyz, R. Kreutz, D. W. Wang, D. Bhella, O. Sagliocco, F. Crea, E. C. Thomson, I. B. McInnes, COVID-19 and the cardiovascular system: implications for risk assessment, diagnosis, and treatment options, Cardiovasc Res (2020).
[19] E. R. Lumbers, S. J. Delforce, K. G. Pringle, G. R. Smith, The Lung, the Heart, the Novel Coronavirus, and the Renin-Angiotensin System; The Need for Clinical Trials, Front Med (Lausanne) 7 (2020) 248.
[20] A. Cai, B. McClafferty, J. Benson, D. Ramgobin, R. Kalayanamitra, Z. Shahid, A. Groff, C. S. Aggarwal, R. Patel, H. Polimera, R. Vunnam, R. Golamari, N. Sahu, D. Bhatt, R. Jain, COVID-19: Catastrophic Cause of Acute Lung Injury, S D Med 73(6) (2020) 252-260.
[21] Y. Y. Zheng, Y. T. Ma, J. Y. Zhang, X. Xie, COVID-19 and the cardiovascular system, Nat Rev Cardiol 17(5) (2020) 259-260.
[22] A. H. J. Danser, M. Epstein, D. Batlle, Renin-Angiotensin System Blockers and the COVID-19 Pandemic: At Present There Is No Evidence to Abandon Renin-Angiotensin System Blockers, Hypertension 75(6) (2020) 1382-1385.
[23] M. A. Sparks, A. South, P. Welling, J. M. Luther, J. Cohen, J. B. Byrd, L. M. Burrell, D. Batlle, L. Tomlinson, V. Bhalla, M. N. Rheault, M. J. Soler, S. Swaminathan, S. Hiremath, Sound Science before Quick Judgement Regarding RAS Blockade in COVID-19, Clin J Am Soc Nephrol 15(5) (2020) 714-716.
[24] C. Bavishi, T. M. Maddox, F. H. Messerli, Coronavirus Disease 2019 (COVID-19) Infection and Renin Angiotensin System Blockers, JAMA Cardiol (2020).
[25] M. Vaduganathan, 0. Vardeny, T. Michel, J. J. V. McMurray, M. A. Pfeffer, S. D. Solomon, Renin-Angiotensin-Aldosterone System Inhibitors in Patients with Covid-19, N Engl J Med 382(17) (2020) 1653-1659.
[26] A. M. South, T. M. Brady, J. T. Flynn, ACE2 (Angiotensin-Converting Enzyme 2), COVID-19, and ACE Inhibitor and Ang II (Angiotensin II) Receptor Blocker Use During the Pandemic: The Pediatric Perspective, Hypertension 76(1) (2020) 16-22.
[27] M. R. Mehra, S. S. Desai, S. Kuy, T. D. Henry, A. N. Patel, Cardiovascular Disease, Drug Therapy, and Mortality in Covid-19, N Engl J Med 382(25) (2020) e102.
[28] A. M. South, D. I. Diz, M. C. Chappell, COVID-19, ACE2, and the cardiovascular consequences, Am J Physiol Heart Circ Physiol 318(5) (2020) H1084-H1090.
[29] R. Sommerstein, M. M. Kochen, F. H. Messerli, C. Grani, Coronavirus Disease 2019 (COVID-19): Do Angiotensin-Converting Enzyme Inhibitors/Angiotensin Receptor Blockers Have a Biphasic Effect?, J Am Heart Assoc 9(7) (2020) e016509.
[30] F. Zhou, T. Yu, R. Du, G. Fan, Y. Liu, Z. Liu, J. Xiang, Y. Wang, B. Song, X. Gu, L. Guan, Y. Wei, H. Li, X. Wu, J. Xu, S. Tu, Y. Zhang, H. Chen, B. Cao, Clinical course and risk factors for mortality of adult inpatients with COVID-19 in Wuhan, China: a retrospective cohort study, Lancet 395(10229) (2020) 1054-1062.
[31] J. Guo, Z. Huang, L. Lin, J. Lv, Coronavirus Disease 2019 (COVID-19) and Cardiovascular Disease: A Viewpoint on the Potential Influence of Angiotensin-Converting Enzyme Inhibitors/Angiotensin Receptor Blockers on Onset and Severity of Severe Acute Respiratory Syndrome Coronavirus 2 Infection, J Am Heart Assoc 9(7) (2020) e016219.
[32] M. Noris, A. Benigni, G. Remuzzi, The case of complement activation in COVID-19 multiorgan impact, Kidney Int (2020).
[33] K. Renu, P. L. Prasanna, A. Valsala Gopalakrishnan, Coronaviruses pathogenesis, comorbidities and multi-organ damage—A review, Life Sci 255 (2020) 117839.
[34] S. Zaim, J. H. Chong, V. Sankaranarayanan, A. Harky, COVID-19 and Multiorgan Response, Curr Probl Cardiol 45(8) (2020) 100618.
[35] H. Bosmuller, S. Traxler, M. Bitzer, H. Haberle, W. Raiser, D. Nann, L. Frauenfeld, A. Vogelsberg, K. Klingel, F. Fend, The evolution of pulmonary pathology in fatal COVID-19 disease: an autopsy study with clinical correlation, Virchows Arch (2020).
[36] T. Menter, J. D. Haslbauer, R. Nienhold, S. Savic, H. Hopfer, N. Deigendesch, S. Frank, D. Turek, N. Willi, H. Pargger, S. Bassetti, J. D. Leuppi, G. Cathomas, M. Tolnay, K. D. Mertz, A. Tzankov, Post-mortem examination of COVID19 patients reveals diffuse alveolar damage with severe capillary congestion and variegated findings of lungs and other organs suggesting vascular dysfunction, Histopathology (2020).
[37] P. Liu, J. Wysocki, P. Serfozo, M. Ye, T. Souma, D. Batlle, J. Jin, A Fluorometric Method of Measuring Carboxypeptidase Activities for Angiotensin II and Apelin-13, Sci Rep 7 (2017) 45473.
[38] A. C. Walls, Y. J. Park, M. A. Tortorici, A. Wall, A. T. McGuire, D. Veesler, Structure, Function, and Antigenicity of the SARS-CoV-2 Spike Glycoprotein, Cell 181(2) (2020) 281-292 e6.
[39] J. Shang, G. Ye, K. Shi, Y. Wan, C. Luo, H. Aihara, Q. Geng, A. Auerbach, F. Li, Structural basis of receptor recognition by SARS-CoV-2, Nature 581(7807) (2020) 221-224.
[40] J. Lan, J. Ge, J. Yu, S. Shan, H. Zhou, S. Fan, Q. Zhang, X. Shi, Q. Wang, L. Zhang, X. Wang, Structure of the SARS-CoV-2 spike receptor-binding domain bound to the ACE2 receptor, Nature 581(7807) (2020) 215-220.
[41] P. Towler, B. Staker, S. G. Prasad, S. Menon, J. Tang, T. Parsons, D. Ryan, M. Fisher, D. Williams, N. A. Dales, M. A. Patane, M. W. Pantoliano, ACE2 X-ray structures reveal a large hinge-bending motion important for inhibitor binding and catalysis, J Biol Chem 279(17) (2004) 17996-8007.
[42] P. Namsolleck, G. N. Moll, Does activation of the protective Renin-Angiotensin System have therapeutic potential in COVID-19?, Mol Med 26(1) (2020) 80.
[43] M. K. Chung, S. Karnik, J. Saef, C. Bergmann, J. Barnard, M. M. Lederman, J. Tilton, F. Cheng, C. V. Harding, J. B. Young, N. Mehta, S. J. Cameron, K. R. McCrae, A. H. Schmaier, J. D. Smith, A. Kalra, S. K. Gebreselassie, G. Thomas, E. S. Hawkins, L. G. Svensson, SARS-CoV-2 and ACE2: The biology and clinical data settling the ARB and ACEI controversy, EBioMedicine 58 (2020) 102907.
[44] R. Sarzani, F. Giulietti, C. Di Pentima, P. Giordano, F. Spannella, Disequilibrium between the classic renin-angiotensin system and its opposing arm in SARS-CoV-2-related lung injury, Am J Physiol Lung Cell Mol Physiol 319(2) (2020) L325-L336.
[45] R. D. Lopes, A. V. S. Macedo, E. S. P. G. M. de Barros, R. J. Moll-Bernardes, A. Feldman, G. D′Andrea Saba Arruda, A. S. de Souza, D. C. de Albuquerque, L. Mazza, M. F. Santos, N. Z. Salvador, C. M. Gibson, C. B. Granger, J. H. Alexander, O. F. de Souza, B. C. investigators, Continuing versus suspending angiotensin-converting enzyme inhibitors and angiotensin receptor blockers: Impact on adverse outcomes in hospitalized patients with severe acute respiratory syndrome coronavirus 2 (SARS-CoV-2)--The BRACE CORONA Trial, Am Heart J 226 (2020) 49-59.
[46] A. Ashkenazi, S. M. Chamow, Immunoadhesins as research tools and therapeutic agents, Curr Opin Immunol 9(2) (1997) 195-200.
[47] S. M. Chamow, A. M. Duliege, A. Ammann, J. O. Kahn, J. D. Allen, J. W. Eichberg, R. A. Byrn, D. J. Capon, R. H. Ward, A. Ashkenazi, CD4 immunoadhesins in anti-HIV therapy: new developments, Int J Cancer Suppl 7 (1992) 69-72.
[48] C. V. Fletcher, J. G. DeVille, P. M. Samson, J. H. Moye, Jr., J. A. Church, H. M. Spiegel, P. Palumbo, T. Fenton, M. E. Smith, B. Graham, J. M. Kraimer, W. T. Shearer, P. S. G. Pediatric Aids Clinical Trials Group, Nonlinear pharmacokinetics of high-dose recombinant fusion protein CD4-IgG2 (PRO 542) observed in HIV-1-infected children, J Allergy Clin Immunol 119(3) (2007) 747-50.
[49] J. M. Jacobson, R. J. Israel, I. Lowy, N. A. Ostrow, L. S. Vassilatos, M. Barish, D. N. Tran, B. M. Sullivan, T. J. Ketas, T. J. O'Neill, K. A. Nagashima, W. Huang, C. J. Petropoulos, J. P. Moore, P. J. Maddon, W. C. Olson, Treatment of advanced human immunodeficiency virus type 1 disease with the viral entry inhibitor PRO 542, Antimicrob Agents Chemother 48(2) (2004) 423-9.
[50] J. M. Jacobson, I. Lowy, C. V. Fletcher, T. J. O'Neill, D. N. Tran, T. J. Ketas, A. Trkola, M. E. Klotman, P. J. Maddon, W. C. Olson, R. J. Israel, Single-dose safety, pharmacology, and antiviral activity of the human immunodeficiency virus (HIV) type 1 entry inhibitor PRO 542 in HIV-infected adults, J Infect Dis 182(1) (2000) 326-9.
[51] W. T. Shearer, R. J. Israel, S. Starr, C. V. Fletcher, D. Wara, M. Rathore, J. Church, J. DeVille, T. Fenton, B. Graham, P. Samson, S. Staprans, J. McNamara, J. Moye, P. J. Maddon, W. C. Olson, Recombinant CD4-IgG2 in human immunodeficiency virus type 1-infected children: phase 1/2 study. The Pediatric AIDS Clinical Trials Group Protocol 351 Study Team, J Infect Dis 182(6) (2000) 1774-9.
[52] S. Fridrich, K. Karmilin, W. Stocker, Handling Metalloproteinases, Curr Protoc Protein Sci 83 (2016) 21 16 1-21 16 20.
[53] F. Namuswe, J. M. Berg, Secondary interactions involving zinc-bound ligands: roles in structural stabilization and macromolecular interactions, J Inorg Biochem 111 (2012) 146-9.
[54] K. A. McCall, C. Huang, C. A. Fierke, Function and mechanism of zinc metalloenzymes, J Nutr 130(5S Suppl) (2000) 1437S-46S.

### Example 3—His345 Mutant of Angiotensin-converting Enzyme 2 (ACE2) Remains Enzymatically Active Against Angiotensin II

Glasgow et al.(1) reported their mutagenesis survey of ACE2-traps for treatment of SARS-CoV-2. The virus invades human cells via host ACE2 receptor, and strategies aimed at disrupting this process are being explored. As compared to monoclonal antibodies and targeted vaccines that are prone to mutational escape of the virus, ACE2-trap strategy has unique advantages and was shown to be efficacious in experimental and clinical tests(2, 3). Current studies, including Glasgow et al., focus on further improvements through alternative designs. These include extending in vivo half-life with an Fc tag(4, 5), increasing Spike-binding affinity via mutagenesis(1, 6), and inactivating ACE2 peptidase activity(l, 4). The Glasgow study combined all three approaches in their lead design(1).

The enzymatic inactivation strategy has two potential benefits: eliminating unwanted cardiovascular side effects attributable to dysregulation of vasoactive hormones, including Angiotensin II (Ang II), and permitting high therapeutic doses without over-supplying ACE2 peptidase activity. It is unfortunate that many researchers, including Glasgow et al., still chose surrogate substrate Mca-APK(Dnp) for measuring ACE2 activity, despite of the apparent distinctions between Mca-APK(Dnp) and Ang II in sequence (FIG. 1A). Glasgow et al. examined two inactivating mutants of ACE2, namely H374N/H378N double mutations of the Zn²⁺-binding residues and H345L mutation at the catalytic center(7). The authors showed loss-of-activity of both constructs against Mca-APK(Dnp). For the reason that the study discovered H374N/H378N being unstable, they incorporate H345L in their lead candidate to have long-action time (via Fc), better Spike-binding (via affinity-enhanced mutation(s)), and being enzymatically inactivated (via H345L mutation).

In parallel to the Glasgow et al. study, our group conducted a mutagenesis survey of ACE2-Fc towards inactivating its activity against physiologic substrates(8). We included mutations intended to disrupt either Zn²⁺-binding (via His374, His378 and Glu402) or substrate catalysis (via Glu145, Arg273, His345, Pro346, Asp368 and His505) with Alanine substitutions of individual residues. We performed both Mca-APK(Dnp) assay and phenylalanine-hydrolysis measurement using Ang II and Apelin-13 as substrates. Our results showed a clear substrate-dependent inactivation among the mutants. Of particular, H345A, P346A and H505A mutants showed complete inactivation of ACE2-Fc against Mca-APK(Dnp) but intact catalysis against Ang II and Apelin-13. With respect to the H345 mutant (mutated to Alanine in our study instead of to Leucine in Glasgow et al.), our results suggest the enzyme can still catalyze Ang II, which is a concern in therapeutic design for SARS-CoV-2.

To ascertain our findings, we repeated the assays and obtained similar results as before (FIG. 1B). In addition, we performed mass spectrometry to measure the generation of Ang 1-7 by ACE2-Fc R273A and H345A. The results confirmed full activity of H345A towards Ang II (FIG. 1C), which argued against the conclusion by Glasgow et al. with regard to the catalytic residue.

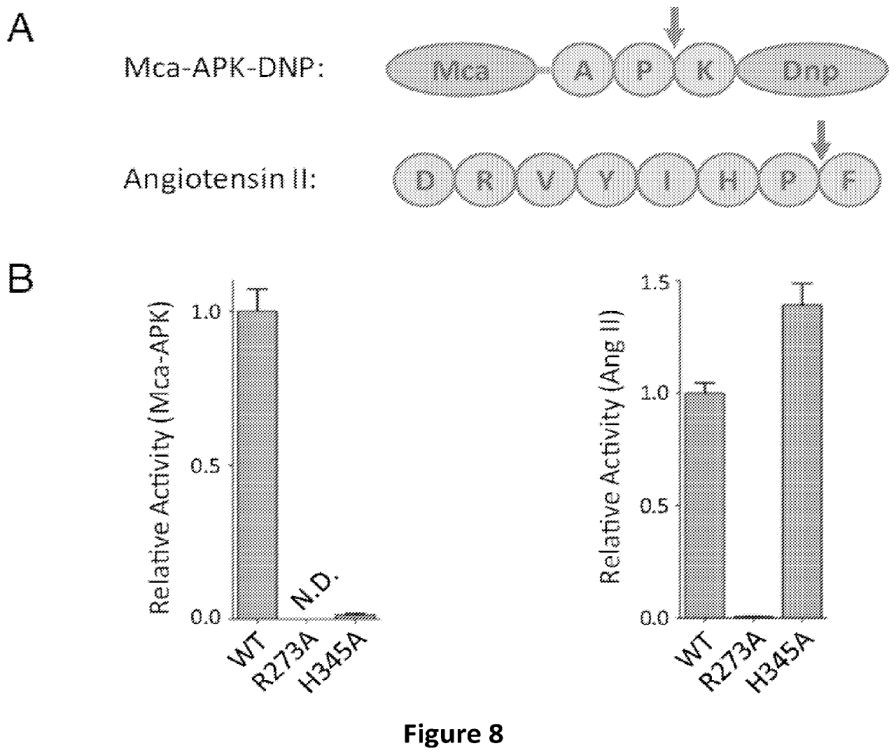

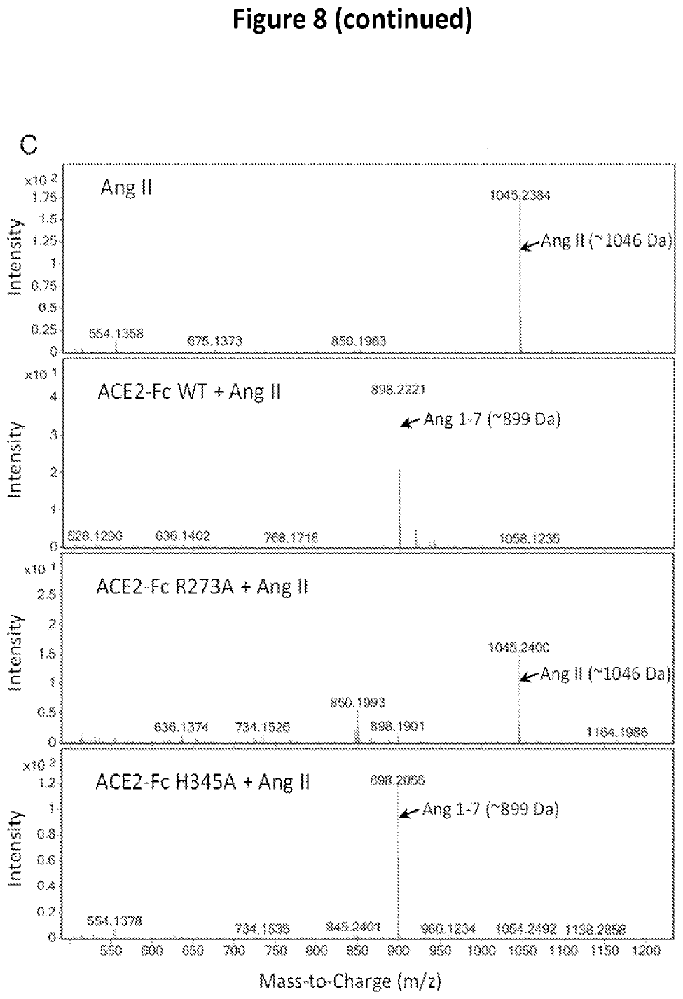

FIG. 8 illustrates the substrate-dependent activities among ACE2 mutants. Surrogate substrate Mca-APK(Dnp) contains the fluorogenic attachments of Methyl 2-Cyanuacrylate (Mca) and Dinitrophenyl (Dnp) to tripeptide sequence Ala-Pro-Lys (APK). Enzymatic cleavage of the peptide bond between Pro-Lys by ACE2 generates fluorescence signal that has been used in measuring ACE2 peptidase activity. However, no physiologic substrates of ACE2 contain this Pro-Lys bond in their sequences. Instead, a C-terminus Pro-Phe dipeptide sequence is shared among many ACE2 substrates, including Ang II, Apelin-13 and Bradykinin. The monocarboxypeptidase ACE2 selectively hydrolyzes these Pro-Phe bonds in its substrates.

In parallel, we performed ACE2 activity assays using either Mca-APK(Dnp) or synthetic Ang II peptide as substrate together with individual variants of ACE2-Fc as the enzyme. By using Mca-APK(Dnp), fluorescence signals at 328 nm excitation and 393 nm emission were recorded. As compared to wild-type (WT) ACE2-Fc, both R273A and H345A showed loss-of-activity in agreement with the findings by Glasgow et al. However, ACE2-Fc enzymatic activity measured by physiologic substrate Ang II showed different results. Via measuring the generation of amino acid phenylalanine from the reaction, R273A remained inactive, whereas H345A showed full activity as compared to wild-type ACE2-Fc.

To further confirm the specificity of the reaction, we conducted mass spectrometry analysis of the peptide(s) generated from the reactions using Ang II peptide as substrate. Without ACE2-Fc in the reaction, Ang II remained intact as a single spike at expected molecular weight of ˜1045 Da. In contrast, following the reaction with wild-type ACE2-Fc, the Ang II signal disappeared, and a new peptide of −898 Da was generated, corresponding to the size of Ang 1-7, suggesting enzymatic cleavage of Ang II and the generation of Ang 1-7 by ACE2. When Ang II was incubated with R273A mutant of ACE2-Fc, no Ang 1-7 signal was observed, indicating loss-of-activity of ACE2-Fc R273A. Following the reaction of Ang II with ACE2-Fc H345A, MS peak corresponding to Ang 1-7 was evident with the complete loss of the original Ang II peak, suggesting full activity of ACE2-Fc H345A mutant towards Ang II.

### REFERENCES

1. A. Glasgow et al., Engineered ACE2 receptor traps potently neutralize SARS-CoV-2. Proc Natl Acad Sci USA 117, 28046-28055 (2020).
2. V. Monteil et al., Inhibition of SARS-CoV-2 Infections in Engineered Human Tissues Using Clinical-Grade Soluble Human ACE2. Cell 181, 905-913 e907 (2020).
3. A. Zoufaly et al., Human recombinant soluble ACE2 in severe COVID-19. Lancet Respir Med 8, 1154-1158 (2020).
4. C. Lei et al., Neutralization of SARS-CoV-2 spike pseudotyped virus by recombinant ACE2-Ig. Nat Commun 11, 2070 (2020).
5. P. Liu et al., Novel ACE2-Fc chimeric fusion provides long-lasting hypertension control and organ protection in mouse models of systemic renin angiotensin system activation. Kidney Int 94, 114-125 (2018).
6. K. K. Chan et al., Engineering human ACE2 to optimize binding to the spike protein of SARS coronavirus 2. Science 369, 1261-1265 (2020).
7. J. L. Guy, R. M. Jackson, H. A. Jensen, N. M. Hooper, A. J. Turner, Identification of critical active-site residues in angiotensin-converting enzyme-2 (ACE2) by site-directed mutagenesis. FEBS J 272, 3512-3520 (2005).
8. P. Liu, X. Xie, L. Gao, J. Jin, Designed variants of ACE2-Fc that decouple anti-SARS-CoV-2 activities from unwanted cardiovascular effects. Int J Biol Macromol 165, 1626-1633 (2020).

### Example 4—Antiviral Effect of ACE2(273)-Fc on SARS-CoV-2

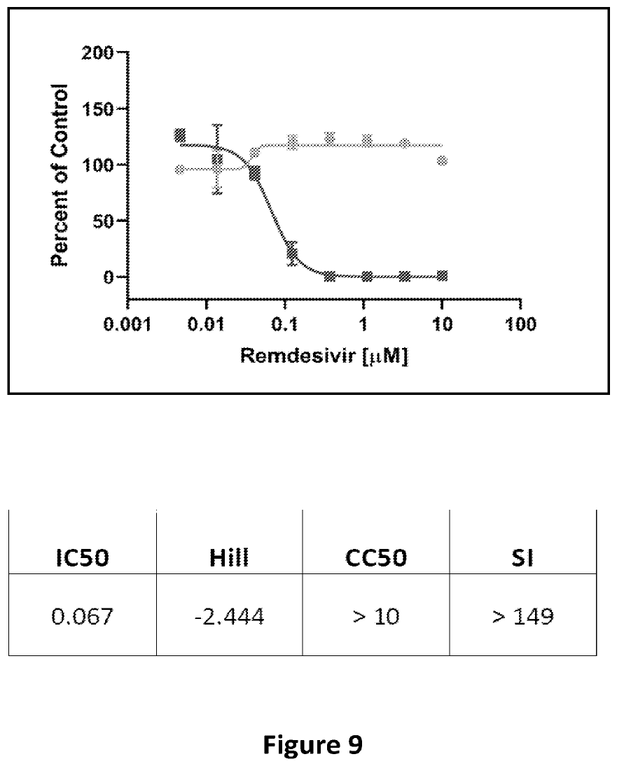

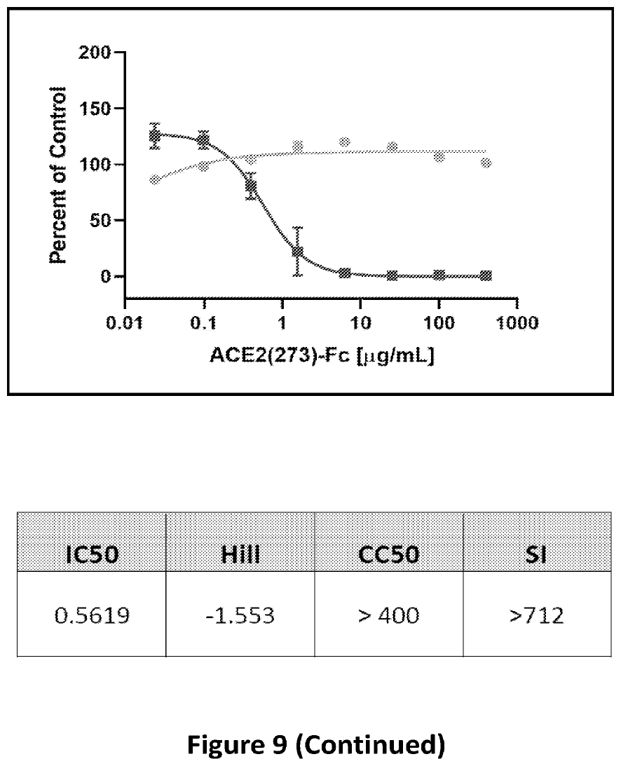

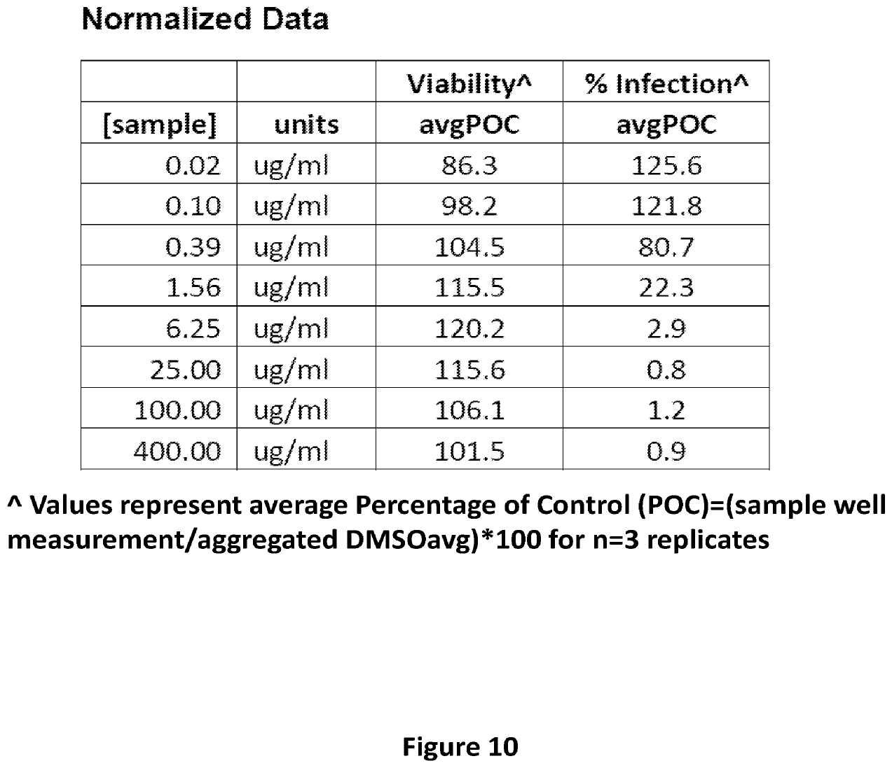

Test 1: Calu3 (ATCC, HTB-55) cells were pretreated with test compounds for 2 hours prior to continuous infection with SARS-CoV-2 (isolate USA WA1/2020) at a MOI=0.5. Forty-eight hours post-infection, cells were fixed, immunostained, and imaged by automated microscopy for infection (dsRNA+ cells/total cell number) and cell number. Sample well data was normalized to aggregated DMSO control wells and plotted versus drug concentration to determine the IC₅₀ and CC50. Results are shown in FIG. 9 and FIG. 10.

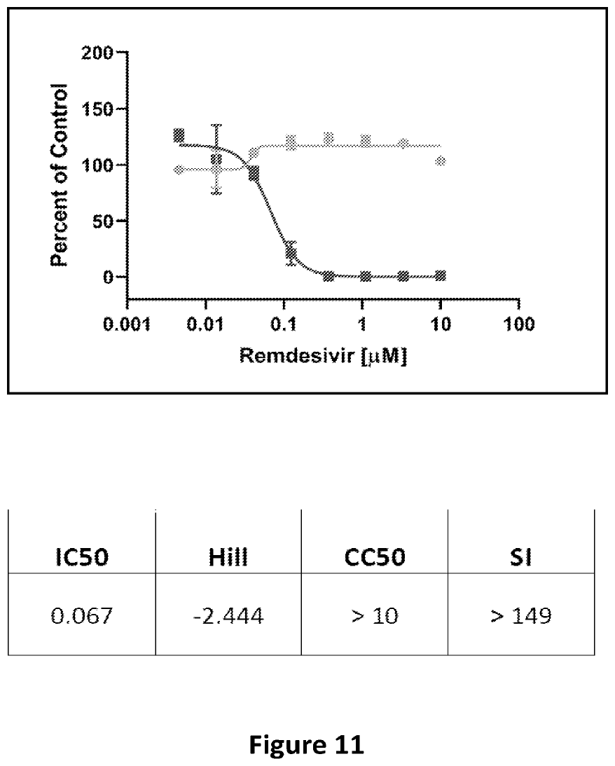

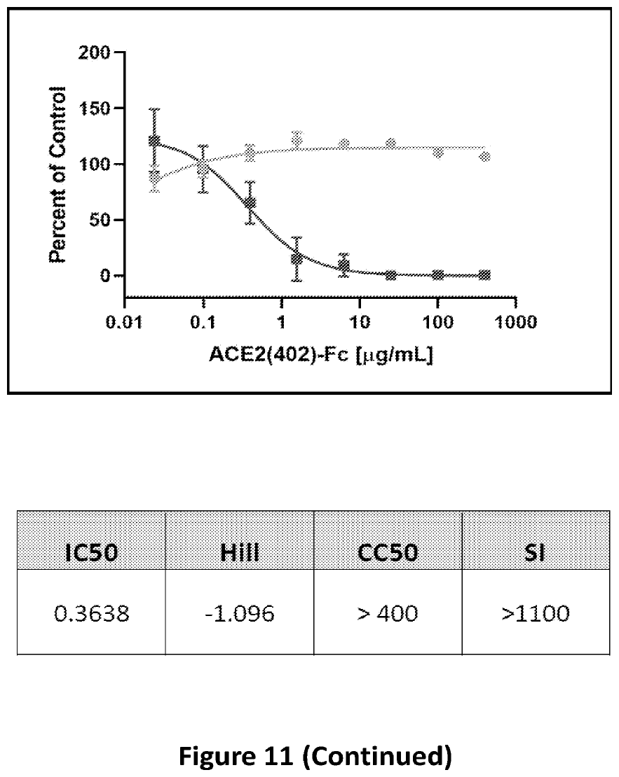

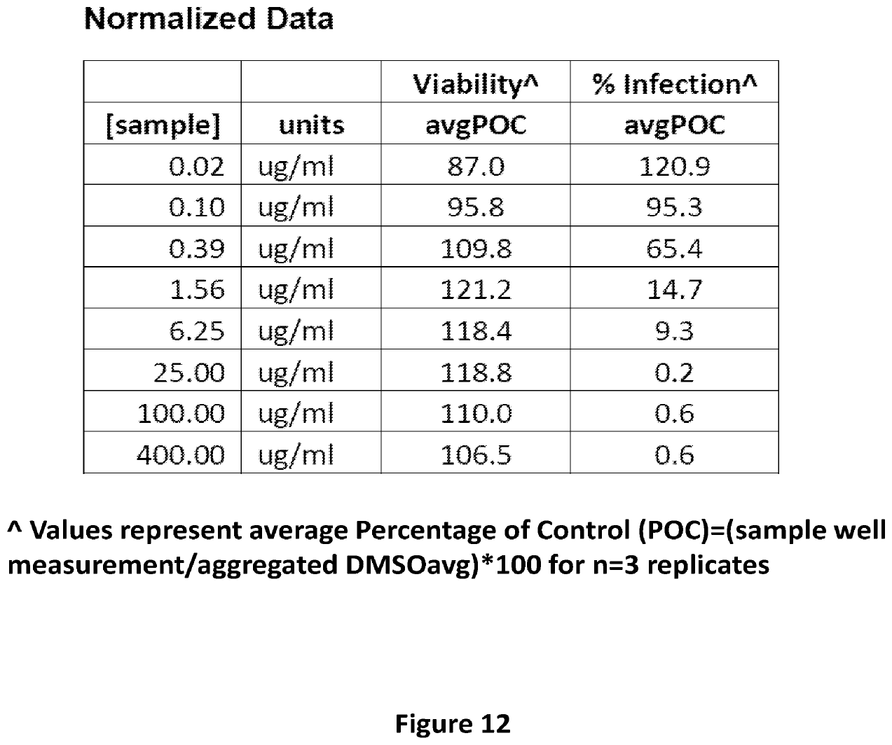

Test 2: Calu3 (ATCC, HTB-55) cells were pretreated with test compounds for 2 hours prior to continuous infection with SARS-CoV-2 (isolate USA WA1/2020) at a MOI=0.5. Forty-eight hours post-infection, cells were fixed, immunostained, and imaged by automated microscopy for infection (dsRNA+ cells/total cell number) and cell number. Sample well data was normalized to aggregated DMSO control wells and plotted versus drug concentration to determine the IC₅₀ and CC₅₀. Results are shown in FIG. 11 and FIG. 12.

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. Any cited references are incorporated by reference herein in their entireties. In the event that there is an inconsistency between a definition of a term in the specification as compared to a definition of the term in a cited reference, the term should be interpreted based on the definition in the specification.

We claim:

1. A fusion protein comprising: (i) at least a portion of the ectodomain of human angiotensin converting enzyme 2 (ACE2) fused directly or via a linking sequence to (ii) at least a portion of the constant region of a human antibody; wherein the fusion protein binds to the spike protein of sudden acute respiratory syndrome coronavirus 2 (SARS-CoV-2) and the fusion protein comprises one or more mutations in ACE2 that result in reduced peptidase activity.

2. The fusion protein of claim 1, wherein the one or more mutations are selected from a mutation at position E145, R273, H345, P346, D368, H374, H378, E402, H505, or combinations thereof.

3. The fusion protein of claim 1, wherein the one or more mutations comprise a mutation at position R273.

4. The fusion protein of claim 1, wherein the one or more mutations comprise the mutation R273A.

5. The fusion protein of claim 1, wherein the one or more mutations comprise a mutation at position H378.

6. The fusion protein of claim 1, wherein the one or more mutations comprise the mutation H378A.

7. The fusion protein of claim 1, wherein the one or more mutations comprise a mutation at position E402.

8. The fusion protein of claim 1, wherein the one or more mutations comprise the mutation E402A.

9. The fusion protein of claim 1, wherein the fusion protein exhibits reduced peptidase activity for Angiotensin II of at least 50% relative to a fusion protein that does not comprise the one or more mutations in ACE2.

10. The recombinant protein of claim 1, wherein the recombinant protein binds to sudden acute respiratory virus coronavirus 2 (SARS-CoV-2), for example, with an equilibrium dissociation constant (K_{d} (M)) of less than about 10⁻⁵, 10⁻⁶, 10⁻⁷, 10⁻⁸, 10⁻⁹, 10⁻¹⁰, 10⁻¹¹, or 10⁻¹².

11. The fusion protein of claim 1, wherein the fusion protein inhibits binding to the spike protein of SARS-CoV-2 and has an EC50 of no more than about 50 ng/ml.

12. The fusion protein of claim 1, wherein the fusion protein inhibits transduction of SARS-CoV-2 into cells that express ACE2 and has an IC₅₀ of no more than about 5 ug/ml.

13. The fusion protein of claim 1, wherein the fusion protein has a half-life 4(1/20 in blood of at least about 40 hours.

14. The fusion protein of claim 1, wherein the portion of the ectodomain of human angiotensin converting enzyme 2 (ACE2) comprises a sequence from amino acid S19 to amino acid F555 of SEQ ID NO:1 and further includes the one or more mutations in ACE2 that result in reduced peptidase activity.

15. The fusion protein of claim 1, wherein the portion of the ectodomain of human angiotensin converting enzyme 2 (ACE2) comprises a sequence from amino acid S19 to amino acid K619 of SEQ ID NO:1 and further includes the one or more mutations in ACE2 that result in reduced peptidase activity.

16. The fusion protein of claim 1, wherein the portion of the ectodomain of human angiotensin converting enzyme 2 (ACE2) comprises a sequence from amino acid S19 to amino acid S740 of SEQ ID NO:1 and further includes the one or more mutations in ACE2 that result in reduced peptidase activity.

17. The fusion protein of claim 1, wherein the portion of the ectodomain of human angiotensin converting enzyme 2 (ACE2) does not comprise and is lacking the amino acid sequence 741-805 of SEQ ID NO:1.

18. The fusion protein of claim 1, wherein the portion of the ectodomain of human angiotensin converting enzyme 2 (ACE2) comprises SEQ ID NO:5, SEQ ID NO:6, SEQ ID NO:7, SEQ ID NO:8, SEQ ID NO:9, SEQ ID NO:10, SEQ ID NO:11, SEQ ID NO:12, or SEQ ID NO:13.

19. The fusion protein of claim 1, wherein the portion of the constant region of the human antibody is portion of a constant region of IgG1.

20. The fusion protein of claim 1, wherein the portion of the constant region of the human antibody comprises the hinge region, CH2 region, and CH3 region.

21. The fusion protein of claim 1, wherein the portion of the constant region of the human antibody comprises SEQ ID NO:14.

22. The fusion protein of claim 1, wherein the fusion protein forms a dimer.

23. The fusion protein of claim 1, wherein the fusion protein dimerizes via an intermolecular cysteine-cysteine disulfide bond formed between the portion of the constant region of the human antibody of one fusion protein and the portion of the constant region of the human antibody of another fusion protein.

24. The fusion protein of claim 1, wherein the fusion protein comprises SEQ ID NO:15, SEQ ID NO:16, SEQ ID NO:17, SEQ ID NO:18, SEQ ID NO:19, SEQ ID NO:20, SEQ ID NO:21, SEQ ID NO:22, and SEQ ID NO:23.

25. The fusion protein of claim 1, wherein the fusion protein comprises a linking sequence comprising 5-15 amino acids selected from glycine, serine, and alanine.

26. A pharmaceutical composition comprising the fusion protein of claim 1 and a suitable pharmaceutical carrier.

27. A method for treating and preventing infection by SARS-CoV-2 in a subject in need thereof, the method comprising administering to the subject the pharmaceutical composition of claim 26.

28. A polynucleotide encoding the fusion protein of claim 1.

29. A human angiotensin converting enzyme 2 (ACE2) protein comprising one or more mutations selected from a mutation at position E145, R273, H345, P346, D368, H374, H378, E402, H505, or combinations thereof.

30. The human ACE2 of claim 29, wherein the mutations are selected from E145A, R273A, H345A, P346A, D368A, H374A, H378A, E402A, H505A, or combinations thereof.

31. The human ACE2 of claim 29, comprising mutations at position R273, H378, E402, or combinations thereof.

32. The human ACE2 of claim 29, comprising mutations at position R273A, H378A, E402A, or combinations thereof.

33. The human ACE2 of claim 29, wherein the human ACE2 is soluble.

34. The human ACE2 of claim 29, wherein the human ACE2 binds to the spike protein of SARS-CoV-2.

35. A pharmaceutical composition comprising the human ACE2 of claim 29 and a suitable pharmaceutical carrier.

36. A method for treating and preventing infection by SARS-CoV-2 in a subject in need thereof, the method comprising administering to the subject the pharmaceutical composition of claim 35.

37. A polynucleotide encoding the human ACE2 of claim 29.
